(19) United States
(12) Patent Application Publication
(10) Pub. No.: US 2019/0084928 A1
(43) Pub. Date: Mar. 21, 2019

(54) METHOD FOR PRODUCING NITROGEN-CONTAINING AROMATIC AMIDE, METHOD FOR PRODUCING PYRROLE-IMIDAZOLE POLYAMIDE, AND COMPOUND

(71) Applicant: National University Corporation Chiba University, Chiba shi, Chiba (JP)

(72) Inventors: Tetsuhiro Nemoto, Chiba-shi, Chiba (JP); Naoki Shiga, Chiba-shi, Chiba (JP); Shihori Takayanagi, Chiba-shi, Chiba (JP); Yuta Suzuki, Chiba-shi, Chiba (JP); Atsushi Kaneda, Chiba-shi, Chiba (JP)

(21) Appl. No.: 16/089,011

(22) PCT Filed: Mar. 2, 2017
(86) PCT No.: PCT/JP2017/008383

§ 371 Date: Sep. 27, 2018

(30) Foreign Application Priority Data
Apr. 1, 2016 (JP) 2016-074677

(51) Int. Cl. C07D 207/34; C07D 233/90; C07D 403/12; C07D 403/14

(52) U.S. Cl. CPC C07D 207/34; C07D 233/90; B01J 27/122; C07D 403/14; C07D 403/12

(57) ABSTRACT

To provide a method for producing a nitrogen-containing aromatic amide that is capable of using a monomer unit obtained under milder reaction condition and uses catalytic amide bond formation, and a method for producing a pyrrole-imidazole polyamide. [1] A method for producing a nitrogen-containing aromatic amide, including reacting a compound 1 represented by the general formula (1) and a compound 2 represented by the general formula (2) in the presence of a transition metal catalyst and a base, so as to provide a compound 3 represented by the general formula (3). [2] A method for producing a pyrrole-imidazole polyamide, including using the compound 3 obtained by the production method according to the item [1]. [3] A compound represented by the general formula (2aa), the general formula (2ab), the general formula (2ac), or the general formula (2ad).

### TECHNICAL FIELD

The present invention relates to a method for producing a nitrogen-containing aromatic amide, a method for producing a pyrrole-imidazole polyamide, and a compound used for the production methods.

### BACKGROUND ART

A pyrrole-imidazole polyamide (which may be hereinafter referred to as a “PIP”) has a function specifically recognizing a base sequence of DNA and binding thereto, so as to repress strongly the transcriptional activity of the target gene. The PIP is a high-order functional middle molecule that is expected to be applied to the medical drug development. The molecular design thereof is derived from the DNA binding capability of distamycin A, which is a natural product having a pyrrole amide trimer structure and exhibiting an anti-cancer activity.

The PIP synthesis method based on peptide condensation developed by Dervan, et al. in the nineties (see NPL 1) has been currently used commonly (see, for example, NPL 2), in which the PIP chain is extended by combining synthesis of pyrrole and an imidazole derivative as monomer units and a solid phase synthesis method. While improved synthesis methods have been reported, there is no essential difference in the peptide condensation utilizing an active ester method (see, for example, NPL 3).

### CITATION LIST

### Non-Patent Literatures

NPL 1: Eldon E Baird, and Peter B. Dervan, Journal of the American Chemical Society, 1996, vol. 118, 6141
NPL 2: David M. Chenoweth, Daniel A, Harki, and Peter B. Dervan, Journal of the American Chemical Society, 2009, vol. 131, 7175
NPL 3: Wu Su, Stephen J. Gray, Ruggero Dondi and Glenn A. Burley. Organic Letters, 2009, vol. 11, 3910

### SUMMARY OF INVENTION

### Technical Problem

In the commonly used synthesis method of a PIP (see, for example, NPL 1), the synthesis is basically performed in the following manner.

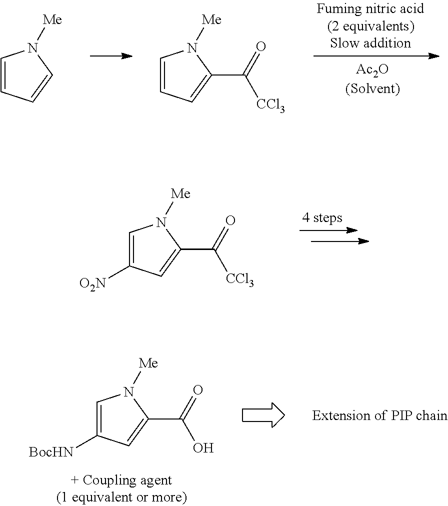

It has been revealed that the commonly used PIP synthesis method has the following problems in the step of preparing a monomer unit and a step of forming an amide bond respectively.

The step of preparing a monomer unit has problems that a large amount of fuming nitric acid is necessarily used in an acetic anhydride solvent, the slow addition thereof carries a high risk and is desired to be avoided where possible from the standpoint of mass production, and the nitration step of the pyrrole derivative has poor reproducibility.

The step of forming an amide bond has a problem that the condensation agent for forming an amide bond connecting the monomer units is necessarily used in an equivalent amount or more, which is not economically effective.

An ideal synthesis method in view of safety and economy may be established in the case where a monomer unit obtained by a synthesis method under milder condition can be used, and the amide bond formation utilizing a condensation agent in an equivalent amount or more can be replaced by catalytic amide bond formation.

Under the circumstances, an object of the present invention is to provide a method for producing a nitrogen-containing aromatic amide that is capable of using a monomer unit obtained under milder reaction condition and uses catalytic amide bond formation, a method for producing a pyrrole-imidazole polyamide, and a novel compound.

### Solution to Problem

The present invention is as follows.

[1] A method for producing a nitrogen-containing aromatic amide, including reacting a compound 1 represented by the general formula (1):

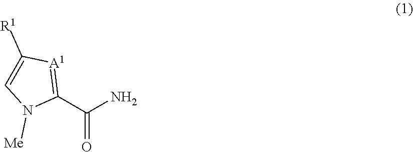

wherein R¹ represents a hydrogen atom or a substituent; and A¹ represents N or CH, and

a compound 2 represented by the general formula (2):

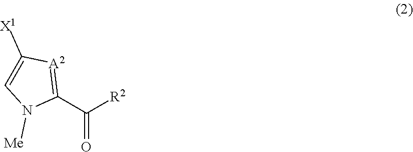

wherein A² represents N or CH; X¹ represents a halogen atom; and R² represents an alkyloxy group having from 1 to 6 carbon atoms or a group represented by the general formula (A): *—NHR^{a}, wherein R^{a} represents a substituent; and * represents a bonding site,
in the presence of a transition metal catalyst and a base, so as to provide a compound 3 represented by the general formula (3):

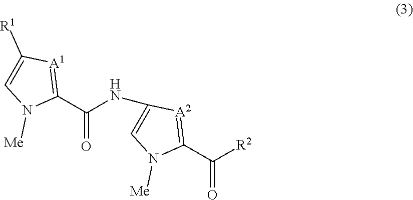

wherein R¹, A¹, A², and R² have the same meanings as in the general formula (1) and the general formula (2).

[2] A method for producing a pyrrole-imidazole polyamide, including using the compound 3 obtained by the production method according to the item [1].

[3] A compound represented by the general formula (2aa), the general formula (2ab), the general formula (2ac), or the general formula (2ad).

### Advantageous Effects of Invention

According to the present invention, a method for producing a nitrogen-containing aromatic amide that is capable of using a monomer unit obtained under milder reaction condition and uses catalytic amide bond formation, a method for producing a pyrrole-imidazole polyamide, and a novel compound can be provided.

### DESCRIPTION OF EMBODIMENTS

The abbreviations used in the description have the following meanings.

PIP: pyrrole-imidazole polyamide

THF: tetrahydrofuran

DMF: N,N-dimethylformamide

DMSO: dimethylsulfoxide

Me group: methyl group

MeCN: acetonitrile

OTf group: triflate group (trifluoromethanesulfonate group)

NIS: N-iodosuccinimide

DMEDA: N,N′-dimethylethylenediamine

Ns group: nosyl group (2-nitrobenzenesulfonyl group)

SES group: trimethylsilylethanesulfonyl group

Ms group: methanesulfonyl group

LHMDS: lithium hexamethyldisilazane

HATU: O-(7-azabenzotriazol-1-yl)-N,N,N′,N′-tetramethyluronium hexafluorophosphate

Boc group: tert-butoxycarbonyl group

BocHN group: N-(tert-butoxycarbonyl)amino group

Ac group: acetyl group

AcHN group: N-(acetyl)amino group

DBH: 1,3-dibromo-5,5-dimethylhydantoin

DMAP: N,N-dimethyl-4-aminopyridine

DIH: 1,3-diiodo-5,5-dimethylhydantoin

The unit “M” means “mol/L”.

### [Method for Producing Nitrogen-Containing Aromatic Amide]

The method for producing a nitrogen-containing aromatic amide of the present invention includes reacting a compound 1 represented by the general formula (1) (which may be hereinafter referred simply to as a “compound 1”) and a compound 2 represented by the general formula (2) (which may be hereinafter referred simply to as a “compound 2”) in the presence of a transition metal catalyst and a base, so as to provide a compound 3 represented by the general formula (3) (which may be hereinafter referred simply to as a “compound 3”) (the reacting step may be hereinafter referred to as a “cross-coupling step”).

As described above, the compound 3 is obtained through cross-coupling reaction using a transition metal catalyst, and thereby a nitrogen-containing aromatic amide can be obtained as the compound 3, using a monomer unit obtained under milder reaction condition through catalytic amide bond formation.

The method may include, before the cross-coupling step, reacting a nitrogen-containing aromatic compound and a halogenating agent, so as to provide the compound 2 (the reacting step may be hereinafter referred to as a “halogenating step”).

The steps will be described in detail below.

### <Cross-Coupling Step>

### [Compound 1]

The compound 1 is represented by the general formula (1):

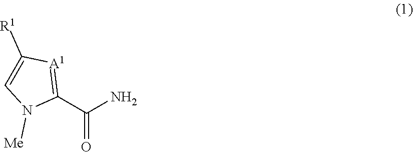

wherein R¹ represents a hydrogen atom or a substituent; and A¹ represents N or CH.

The substituent represented by R¹ may be a BocHN group, an AcHN group, or a group represented by the general formula (B):

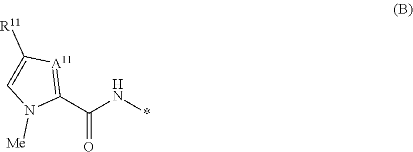

wherein R¹¹ represents a hydrogen atom, a BocHN group, an AcHN group, or a group represented by the general formula (B′):

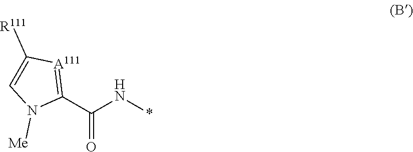

wherein R¹¹¹ represents a hydrogen atom, a BocHN group or an AcHN group; A¹¹¹ represents N or CH; and * represents a bonding site;

A¹¹ represents N or CH; and * represents a bonding site.

R¹¹ preferably represents a hydrogen atom.

R¹¹¹ preferably represents a hydrogen atom.

R¹ preferably represents a hydrogen atom, a BocHN group, an AcHN group, or a group represented by the general formula (B), and more preferably a hydrogen atom or a group represented by the general formula (B).

### [Compound 2]

The compound 2 is represented by the general formula (2):

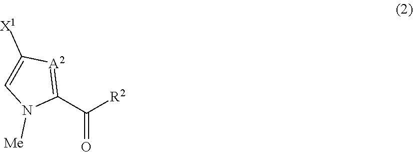

wherein A² represents N or CH; X¹ represents a halogen atom; and R² represents an alkyloxy group having from 1 to 6 carbon atoms or a group represented by the general formula (A): *—NHR^{a}, wherein R^{a} represents a substituent; and * represents a bonding site.

A² preferably represents CH from the standpoint of the enhancement of the yield.

X¹ represents, for example, a chlorine atom, a bromine atom, or an iodine atom, and preferably a bromine atom or an iodine atom, and is preferably an iodine atom from the standpoint of the enhancement of the yield.

In the case where R² represents an alkyloxy group, the alkyloxy group is preferably an alkyloxy group having from 1 to 3 carbon atoms. Examples of the alkyloxy group represented by R² include a methoxy group, an ethoxy group, and a hexyloxy group, and a methoxy group is preferred.

R^{a} in the group represented by the general formula (A) may represent an alkyl group having from 1 to 6 carbon atoms, an aralkyl group having from 7 to 20 carbon atoms, an aminoalkyl group having from 1 to 6 carbon atoms, or a group represented by the general formula (C):

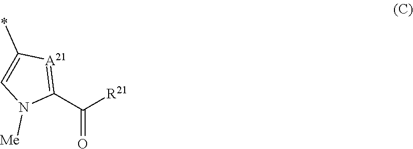

wherein A²¹ represents N or CH; and R²¹ represents an alkyloxy group having from 1 to 6 carbon atoms or a group represented by the general formula (A′): *—NHR^{a′}, wherein R^{a′} represents a substituent; and * represents a bonding site. The preferred examples for R²¹ may be the same as the preferred examples for R².

Examples of the alkyl group represented by R^{a} include a methyl group and an ethyl group.

Examples of the aralkyl group represented by R^{a} include a benzyl group.

Examples of the aminoalkyl group represented by R^{a} include an N,N-dialkylaminoalkyl group and an aminoalkyl group.

Among these, R^{a} preferably represents a group represented by the general formula (C).

R^{a′} in the group represented by the general formula (A′) may represent an alkyl group having from 1 to 6 carbon atoms, an aralkyl group having from 7 to 20 carbon atoms, an aminoalkyl group having from 1 to 6 carbon atoms, or a group represented by the general formula (C′):

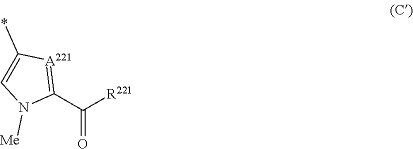

wherein A²²¹ represents N or CH; and R²²¹ represents an alkyloxy group having from 1 to 6 carbon atoms or a group represented by the general formula (A″): *—NHR^{a″}, wherein R^{a″} represents an alkyl group having from 1 to 6 carbon atoms, an aralkyl group having from 7 to 20 carbon atoms, or an aminoalkyl group having from 1 to 6 carbon atoms; and * represents a bonding site.

The preferred examples for R²²¹ may be the same as the preferred examples for R².

Examples of the alkyl group represented by R^{a′} and R^{a″} include a methyl group and an ethyl group.

Examples of the aralkyl group represented by R^{a′} and R^{a″} include a benzyl group.

Examples of the aminoalkyl group represented by R^{a′} and R^{a″} include an N,N-dialkylaminoalkyl group and an aminoalkyl group.

### [Transition Metal Catalyst]

Examples of the transition metal catalyst include a catalyst containing at least one selected from a copper catalyst, a palladium catalyst, a nickel catalyst, and a platinum catalyst.

The copper catalyst is preferably a monovalent copper catalyst, more preferably a copper(I) halide, and further preferably copper(I) iodide.

Examples of the palladium catalyst include tris(dibenzylideneacetone) dipalladium (Pd₂(dba)₃), bis(dibenzylideneacetone) palladium (0) (Pd(dba)₂), tetrakis(triphenylphosphine) palladium(0), bis(allylchloropalladium(II)), palladium(II) chloride, and palladium(II) acetate.

Examples of the nickel catalyst include bis(1,5-cyclooctadiene) nickel(0) (Ni(cod)₂) and bis(allylchloronickel(II)).

Examples of the platinum catalyst include bis(1,5-cyclooctadiene) platinum(0) (Pt(cod)₂).

Among these, the transition metal catalyst is preferably a copper catalyst, more preferably a monovalent copper catalyst, further preferably a copper(I) halide, and still further preferably copper(I) iodide, from the standpoint of the enhancement of the yield of the compound 3.

The amount of the transition metal catalyst used is preferably from 1 to 20% by mol, and more preferably from 1 to 15% by mol, in terms of the transition metal contained in the transition metal catalyst based on the molar number of the compound 2.

### [Ligand]

A ligand is preferably used in addition to the transition metal catalyst from the standpoint of the enhancement of the yield of the compound 3.

Examples of the ligand include a nitrogen-containing ligand and a phosphine ligand.

The ligand may be a monodentate ligand, a bidentate ligand, or a multidentate ligand that is more than bidentate, and a bidentate ligand is preferred.

Examples of the nitrogen-containing ligand include N,N′-dimethylethylenediamine, N,N,N′,N′-tetramethylethylene diamine, ethylenediamine, 2,2′-bipyridine, and 1,10-phenanthroline.

Examples of the phosphine ligand include triphenylphosphine, tricyclohexylphosphine, 2,2′-bis(diphenylphosphino)-1,1′-binaphthyl, 2-dicyclohexylphosphino-2′,4′,6′-trisisopropylbiphenyl, and 4,5-bis(diphenylphosphino)-9,9-dimethylxanthene.

In the case where a copper catalyst is used, the ligand is preferably a bidentate ligand, more preferably a nitrogen-containing ligand, further preferably at least one selected from N,N′-dimethylethylenediamine, N,N,N′,N′-tetramethylethylene diamine, and ethylenediamine, and still further preferably N,N′-dimethylethylenediamine.

The amount of the ligand used is preferably from 2 to 6 equivalents, and more preferably from 3 to 5 equivalents, based on the transition metal contained in the transition metal catalyst.

The equivalent herein means the molar number of the coordination site (such as a phosphine site or an amine site) per 1 mol of the transition metal.

In the case where a bidentate ligand is used, the molar ratio of the bidentate ligand with respect to the transition metal contained in the transition metal catalyst (bidentate ligand/transition metal) is preferably 1 or more, more preferably 2 or more, further preferably 3 or more, and still further preferably 3.5 or more, and is preferably 12 or less, more preferably 10 or less, further preferably 8 or less, still further preferably 6 or less, and still more further preferably 5 or less.

### [Base]

Examples of the base include sodium phosphate (Na₃PO₄), potassium phosphate (K₃PO₄), sodium carbonate (Na₂CO₃), potassium carbonate (K₂CO₃), rubidium carbonate (Rb₂CO₃), cesium carbonate (Cs₂CO₃), sodium acetate (NaOCOCH₃), and potassium acetate (KOCOCH₃).

Among these, potassium phosphate and cesium carbonate are preferred, potassium phosphate is preferred in the case where A¹ and A² are CH, and cesium carbonate is preferred in the case where A¹ is N, and A² is CH.

The amount of the base used is preferably from 1 to 4 equivalent, and more preferably from 2 to 3 equivalent, based on the compound 2.

### [Solvent]

Examples of a solvent used in the cross-coupling step include 1,4-dioxane, tetrahydropyran, THF, toluene, and xylene, and among these, 1,4-dioxane and toluene are preferred, and 1,4-dioxane is more preferred.

The concentration of the compound 2 in the solvent is preferably from 0.1 to 3 M, more preferably from 0.2 to 2 M, and further preferably 0.3 to 1.5 M.

### [Reaction Condition]

The reaction temperature is not particularly limited, and is, for example, from room temperature (25° C.) to 200° C., preferably from 70 to 180° C., more preferably from 80 to 150° C., and further preferably from 90° C. to 130° C.

The reaction time is not particularly limited, and is, for example, from 1 to 48 hours, preferably from 6 to 36 hours, more preferably from 12 to 30 hours, and further preferably from 20 to 28 hours.

After the reaction, the compound can be purified by a known method, such as Celite filtration, silica gel column chromatography, and organic solvent extraction.

The compound 3 represented by the general formula (3) is obtained through the cross-coupling step:

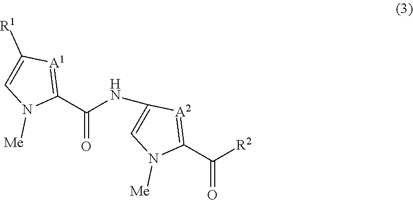

wherein R¹, A¹, A², and R² have the same meanings as in the general formula (1) and the general formula (2).

### (Py-Py)

In the method for producing a nitrogen-containing aromatic amide of the present invention, it is possible that the compound 1 is a compound 1′ represented by the general formula (1′):

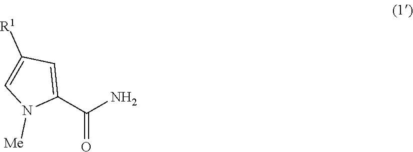

wherein R¹ has the same meaning as in R¹ in the general formula (1), and the compound 2 is a compound 2′ represented by the general formula (2′):

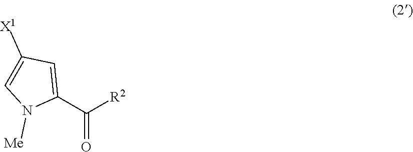

wherein X¹ and R² have the same meanings as in the general formula (2). In this case, the following embodiments are preferred from the standpoint of the enhancement of the yield of the compound 3.

The transition metal catalyst is preferably a monovalent copper catalyst, more preferably a copper(I) halide, and further preferably copper(I) iodide.

The ligand is preferably a bidentate ligand, more preferably a nitrogen-containing ligand, further preferably at least one selected from N,N′-dimethylethylenediamine, N,N,N′,N′-tetramethylethylene diamine, and ethylenediamine, and still further preferably N,N′-dimethylethylenediamine.

The molar ratio of the bidentate ligand with respect to the transition metal contained in the transition metal catalyst (bidentate ligand/transition metal) is preferably 1 or more, and more preferably 2 or more, and is preferably 12 or less, more preferably 10 or less, further preferably 8 or less, still further preferably 6 or less, and still more further preferably 5 or less.

The base is preferably potassium phosphate or cesium carbonate, and more preferably potassium phosphate.

The solvent is preferably 1,4-dioxane or toluene, and more preferably 1,4-dioxane.

The concentration of the compound 2 in the solvent is preferably from 0.6 to 1.5 M, and more preferably from 0.8 to 1.2 M.

### (Py-Im)

In the method for producing a nitrogen-containing aromatic amide of the present invention, it is possible that the compound 1 is a compound 1′ represented by the general formula (1′):

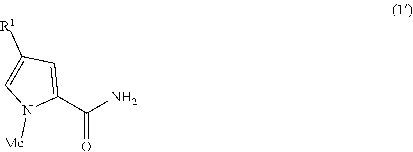

wherein R¹ has the same meaning as in R¹ in the general formula (1), and the compound 2 is a compound 2″ represented by the general formula (2″):

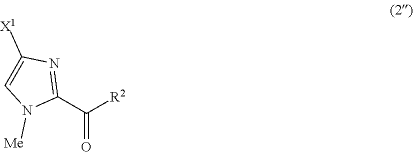

wherein X¹ and R² have the same meanings as in the general formula (2). In this case, the following embodiments are preferred from the standpoint of the improvement of the yield of the compound 3.

R² preferably represents a group represented by the general formula (A): *—NHR^{a}, wherein R^{a} represents a substituent; and * represents a bonding site.

R^{a} preferably represents an aralkyl group having from 7 to 20 carbon atoms, and more preferably a benzyl group.

X¹ preferably represents a bromine atom or an iodine atom, and more preferably an iodine atom.

The transition metal catalyst is preferably a monovalent copper catalyst, more preferably a copper(I) halide, and further preferably copper(I) iodide.

The ligand is preferably a bidentate ligand, more preferably a nitrogen-containing ligand, further preferably at least one selected from N,N′-dimethylethylenediamine, N,N,N′,N′-tetramethylethylene diamine, and ethylenediamine, and still further preferably N,N′-dimethylethylenediamine.

The molar ratio of the bidentate ligand with respect to the transition metal contained in the transition metal catalyst (bidentate ligand/transition metal) is preferably 2 or more, more preferably 3 or more, and further preferably 3.5 or more, and is preferably 6 or less, more preferably 5 or less, and further preferably 4.5 or less.

The base is preferably potassium phosphate or cesium carbonate.

The solvent is preferably 1,4-dioxane.

The concentration of the compound 2 in the solvent is preferably from 0.1 to 1.5 M, more preferably from 0.2 to 0.8 M, and further preferably from 0.2 to 0.6 M.

### (Im-Py)

In the method for producing a nitrogen-containing aromatic amide of the present invention, it is possible that the compound 1 is a compound 1″ represented by the general formula (1″):

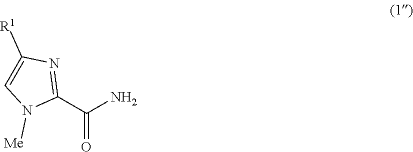

wherein R¹ has the same meaning as in R¹ in the general formula (1), and the compound 2 is a compound 2′ represented by the general formula (2′):

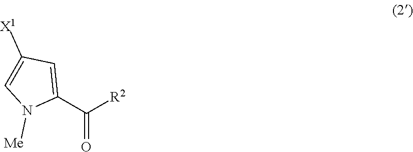

wherein X¹ and R² have the same meanings as in the general formula (2). In this case, the following embodiments are preferred from the standpoint of the enhancement of the yield of the compound 3.

The transition metal catalyst is preferably a monovalent copper catalyst, more preferably a copper(I) halide, and further preferably copper(I) iodide.

The ligand is preferably a bidentate ligand, more preferably a nitrogen-containing ligand, further preferably at least one selected from N,N′-dimethylethylenethamine, N,N,N′,N′-tetramethylethylene diamine, and ethylenediamine, and still further preferably N,N′-dimethylethylenediamine.

The molar ratio of the bidentate ligand with respect to the transition metal contained in the transition metal catalyst (bidentate ligand/transition metal) is preferably 2 or more, more preferably 3 or more, and further preferably 3.5 or more, and is preferably 6 or less, more preferably 5 or less, and further preferably 4.5 or less.

The base is preferably potassium phosphate or cesium carbonate, and more preferably cesium carbonate.

The solvent is preferably 1,4-dioxane.

### (Im-Im)

In the method for producing a nitrogen-containing aromatic amide of the present invention, it is possible that the compound 1 is a compound 1″ represented by the general formula (1″):

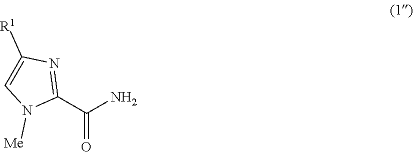

wherein R¹ has the same meaning as in R¹ in the general formula (1), and the compound 2 is a compound 2″ represented by the general formula (2″):

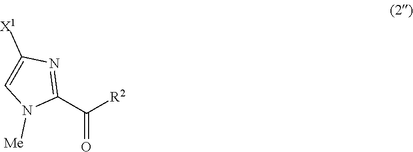

wherein X¹ and R² have the same meanings as in the general formula (2). In this case, the following embodiments are preferred from the standpoint of the enhancement of the yield of the compound 3.

R² preferably represents a group represented by the general formula (A): *—NHR^{a}, wherein R^{a} represents a substituent; and * represents a bonding site.

R^{a} preferably represents an aralkyl group having from 7 to 20 carbon atoms, and more preferably a benzyl group.

X¹ preferably represents a bromine atom or an iodine atom, and more preferably an iodine atom.

The transition metal catalyst is preferably a monovalent copper catalyst, more preferably a copper(I) halide, and further preferably copper(I) iodide.

The ligand is preferably a bidentate ligand, more preferably a nitrogen-containing ligand, further preferably at least one selected from N,N′-dimethylethylenethamine, N,N,N′,N′-tetramethylethylene diamine, and ethylenediamine, and still further preferably N,N′-dimethylethylenediamine.

The molar ratio of the bidentate ligand with respect to the transition metal contained in the transition metal catalyst (bidentate ligand/transition metal) is preferably 2 or more, more preferably 3 or more, and further preferably 3.5 or more, and is preferably 6 or less, more preferably 5 or less, and further preferably 4.5 or less.

The base is preferably potassium phosphate or cesium carbonate.

The solvent is preferably 1,4-dioxane.

The concentration of the compound 2 in the solvent is preferably from 0.1 to 1.5 M, more preferably from 0.2 to 0.8 M, and further preferably from 0.2 to 0.6 M.

### [Halogenating Step]

The method for producing a nitrogen-containing aromatic amide of the present invention preferably further includes a halogenating step before the cross-coupling step.

[Step 1a]

In the case where the compound 2 is a compound 2a represented by the general formula (2a);

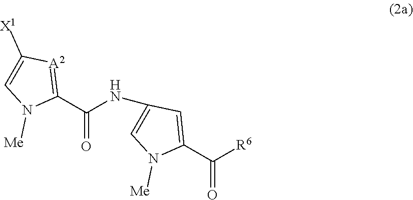

wherein X¹ represents a halogen atom; A² represents N or CH; and R⁶ represents an alkyloxy group having from 1 to 6 carbon atoms,

the halogenating step preferably includes;

reacting a compound represented by the general formula (2a-1) (which may be hereinafter referred simply to as a “compound 2a-1”) and a halogenating agent, so as to provide a compound represented by the general formula (2a-2) (which may be hereinafter referred simply to as a “compound 2a-2”) (the reacting step may be hereinafter referred to as a “step 1a-1”), and

reacting the compound represented by the general formula (2a-2) and an alcohol having from 1 to 6 carbon atoms in the presence of a base, so as to provide the compound 2a represented by the general formula (2a) (the reacting step may be hereinafter referred to as a “step 1a-2”),

from the standpoint that the compound 2a represented by the general formula (2a) is obtained with a high yield and a high selectivity.

The compound 2a-1 has an electron attracting group as the ester substituent R⁷, and thereby the halogenation of the nitrogen-containing aromatic ring with the halogenating agent may proceed regioselectively, resulting in the enhancement of the yield of the compound 2a-2.

Furthermore, after the halogenation reaction, R⁷ in the general formula (2a-1) can be easily substituted, providing the compound 2a with a high yield.

[Step 1a-1]

The compound 2a-1 is represented by the general formula (2a-1):

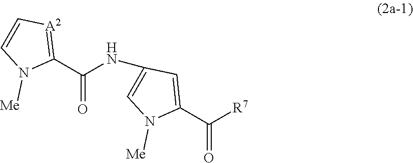

wherein A² represents N or CH; and R⁷ represents a fluorinated aryloxy group, a fluorinated alkyloxy group having from 1 to 6 carbon atoms, or a nitrophenyloxy group.

R⁷ preferably represents a fluorinated aryloxy group, more preferably a fluorinated aryloxy group having from 6 to 12 carbon atoms, and further preferably a fluorinated phenyloxy group.

Examples of the fluorinated aryloxy group include a pentafluorophenyloxy group, a tetrafluorophenyloxy group, a trifluorophenyloxy group, and a heptafluoronaphthyloxy group, and among these, a pentafluorophenyloxy group is preferred.

The compound 2a-1 can be obtained by a known method, and for example, can be obtained in such a manner that an alkyl ester compound of the compound 2a-1 is converted to a carboxylic acid through reaction with a base, such as sodium hydroxide, and subjected to ester exchange with a compound represented by the general formula: CF₃C(O)R⁷ in the presence of a base catalyst.

Examples of the halogenating agent include an N-halosuccinimide, a halogen, such as iodine (I₂) and bromine (Br₂), bis(2,4,6-trimethylpyridine) iodonium salt, bis(2,4,6-trimethylpyridine) bromonium salt, 1,3-diiodo-5,5-dimethylhydantoin, and 1,3-dibromo-5,5-dimethylhydantoin.

Among these, an N-halosuccinimide is preferred.

While the N-halosuccinimide may be appropriately selected depending on the halogen atom to be introduced, examples thereof include N-chlorosuccinimide, N-bromosuccinimide, and N-iodosuccinimide, and N-iodosuccinimide is preferred.

The amount of the N-halosuccinimide is preferably from 1.0 to 4.0 equivalents, and more preferably from 1.0 to 2.0 equivalents, based on the compound 2a-1.

In the step 1a-1, an acid catalyst may be used.

The acid catalyst is preferably a Lewis acid catalyst, more preferably an earth metal compound, and further preferably an indium compound. The earth metal referred herein is a generic term for aluminum, gallium, indium, and thallium.

Specific examples of the acid catalyst include indium tris(trifluoromethyl sulfonate), indium bromide (InBr₃), silver (trifluoromethyl sulfonate), ytterbium tris(trifluoromethyl sulfonate), and trimethylsilyltrifluoromethyl sulfonate (TMSOTf).

The amount of the acid catalyst used is preferably from 1 to 20% by mol, more preferably from 3 to 18% by mol, and further preferably from 5 to 15% by mol, based on the compound 2a-1.

Examples of an organic solvent used in the step 1a-1 include acetonitrile, acetone, THF, DMF, and DMSO, and acetonitrile is preferred.

The concentration of the compound 2a-1 in the solvent is preferably from 0.1 to 3.0 M, more preferably from 0.2 to 2.0 M, and further preferably from 0.3 to 1.5 M.

### [Reaction Condition]

The reaction is preferably performed by adding the halogenating agent to an organic solvent solution of the compound 2a-1.

The temperature in the addition of the halogenating agent is preferably from −80 to 3° C., more preferably from −40 to 3° C., and further preferably from −10 to 3° C., from the standpoint of the enhancement of the yield of the target compound.

After the addition, the reaction is preferably performed with rise of the temperature, and the temperature after the rise of the temperature is, for example, from 10 to 50° C., and preferably from 10 to 45° C., more preferably from 10 to 40° C., and further preferably from 10° C. to room temperature (25° C.).

The reaction time is not particularly limited, and is, for example, from 0.5 to 24 hours, preferably from 1 to 12 hours, and more preferably from 1 to 6 hours.

After the reaction, the compound can be purified by a known method, such as silica gel column chromatography and organic solvent extraction.

A compound represented by the general formula (2a-2) can be obtained through the aforementioned procedure:

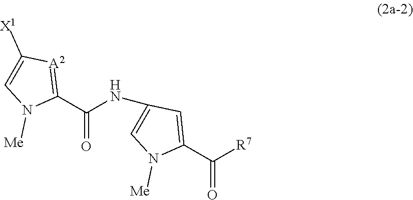

wherein X¹ represents a halogen atom; and A² and R⁷ have the same meanings as in the general formula (2a-1).
[Step 1a-2]

The number of carbon atoms of the alcohol used in the step 1a-2 is preferably from 1 to 4, more preferably from 1 to 3, and further preferably 1 or 2.

Examples of the alcohol include methanol, ethanol, n-propanol, isopropanol, n-butanol, isobutanol, tert-butanol, and hexanol.

Examples of the base used in the step 1a-2 include sodium hydride, potassium hydride, sodium methoxide, potassium methoxide, sodium ethoxide, potassium ethoxide, and potassium tert-butoxide, and among these, sodium hydride is preferred.

The amount of the base used is preferably from 1.0 to 3.0 equivalents, more preferably from 1.0 to 2.0 equivalents, and further preferably from 1.2 to 1.8 equivalents, based on the compound 2a-2.

Examples of an organic solvent used in the step 1a-2 include acetonitrile, acetone, THF, DMF, and DMSO, and THF is preferred.

The organic solvent used in the step 1a-2 is preferably a mixed solvent of an alcohol solvent and THF.

The concentration of the compound 2a-2 in the solvent is preferably from 0.1 to 3.0 M, more preferably from 0.2 to 2.0 M, and further preferably from 0.3 to 1.5 M.

### [Reaction Condition]

The reaction is preferably performed by adding the base to an organic solvent solution of the compound 2a-2.

The temperature in the addition of the base is preferably from −80 to 3° C., more preferably from −40 to 3° C., and further preferably from −10 to 3° C., from the standpoint of the enhancement of the yield of the target compound.

After the addition, the reaction is preferably performed with rise of the temperature, and the temperature after the rise of the temperature is, for example, from 10 to 50° C., and preferably from 10 to 45° C., more preferably from 10 to 40° C., and further preferably from 10° C. to room temperature (25° C.).

The reaction time is not particularly limited, and is, for example, from 1 to 60 minutes, preferably from 5 to 30 minutes, and more preferably from 10 to 20 minutes.

After the reaction, the compound can be purified by a known method, such as silica gel column chromatography and organic solvent extraction.

The compound 2a can be obtained through the aforementioned procedure.

[Step 1b]

In the case where the compound 2 is the compound 2a represented by the general formula (2a),

the halogenating step preferably includes:

reacting a compound represented by the general formula (2a-3) (which may be hereinafter referred simply to as a “compound 2a-3”) and a halogenating agent, so as to provide a compound represented by the general formula (2a-4) (which may be hereinafter referred simply to as a “compound 2a-4”) (the reacting step may be hereinafter referred to as a “step 1b-1”), and

substituting a protective group of the compound represented by the general formula (2a-4) with a hydrogen atom, so as to provide the compound represented by the general formula (2a) (the substituting step may be hereinafter referred to as a “step 1b-2”),

from the standpoint that the compound 2a represented by the general formula (2a) is obtained with a high selectivity.

The compound 2a-3 has an electron attracting protective group such as a nosyl group as R⁸, and thereby the halogenation of the nitrogen-containing aromatic ring with the halogenating agent may proceed regioselectively, resulting in the enhancement of the yield of the compound 2a-4, and the target compound 2a can be obtained with a high yield in the subsequent reaction with a thiol compound.

[Step 1b-1]

The compound 2a-3 is a compound represented by the general formula (2a-3):

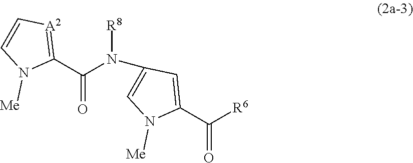

wherein A² represents N or CH; R⁶ represents an alkyloxy group having from 1 to 6 carbon atoms; and R⁸ represents a protective group selected from the group consisting of a nosyl group, a trimethylsilylethanesulfonyl group, a methanesulfonyl group, a trifluoroacetyl group, and a trifluoromethanesulfonyl group.

The compound 2a-3 can enhance the regioselectivity of the halogenation owing to the electron attracting protective group introduced as R⁸.

R⁸ preferably represents a nosyl group from the standpoint of the enhancement of the selectivity of the halogenation reaction and the standpoint of the detachment of the substituent after the halogenation reaction.

The preferred examples of the halogenating agent and the amount thereof used in the step 1b-1 may be the same as exemplified in the step 1a-1. An acid catalyst may be used in the step 1b-1, and the preferred examples of the acid catalyst and the amount thereof used may be the same as exemplified in the step 1a-1.

Furthermore, the preferred examples of the organic solvent, the concentration of the compound in the organic solvent, and the reaction condition may be the same as exemplified in the step 1a-1.

A compound represented by the general formula (2a-4) can be obtained through the aforementioned procedure:

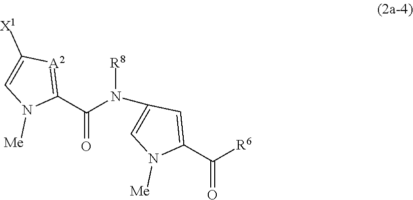

wherein X¹ represents a halogen atom; and A², R⁶ and R⁸ have the same meanings as in the general formula (2a-3).
[Step 1b-2]

The substitution of the protective group of the compound represented by the general formula (2a-4) with a hydrogen atom can be performed by a known method corresponding to the kind of the protective group.

In the case where the protective group is a nosyl group, the step 1b-2 is preferably reacting the compound represented by the general formula (2a-4) and a thiol compound in the presence of a base, so as to provide the compound 2a represented by the general formula (2a).

Examples of the thiol compound include thiophenol and an alkylthiol having from 1 to 20 carbon atoms.

The amount of the thiol compound used is preferably from 1.0 to 8.0 equivalents, more preferably from 2.0 to 6.0 equivalents, and further preferably from 3.0 to 5.0 equivalents, based on the compound 2a-4.

Examples of the base include sodium phosphate (Na₃PO₄), potassium phosphate (K₃PO₄), sodium carbonate (Na₂CO₃), potassium carbonate (K₂CO₃), rubidium carbonate (Rb₂CO₃), cesium carbonate (Cs₂CO₃), sodium acetate (NaOCOCH₃), and potassium acetate (KOCOCH₃).

Among these, potassium carbonate is preferred.

The amount of the base used is preferably from 1.0 to 9.0 equivalents, more preferably from 3.0 to 7.0 equivalents, and further preferably from 4.0 to 6.0 equivalents, based on the compound 2a-4.

The reaction temperature is not particularly limited, and is, for example, from room temperature (25° C.) to 200° C., preferably from room temperature (25° C.) to 150° C., more preferably from 30 to 100° C., and further preferably from 40° C. to 80° C.

The reaction time is not particularly limited, and is, for example, from 0.5 to 24 hours, preferably from 1 to 12 hours, more preferably from 1 to 6 hours, and further preferably from 2 to 4 hours.

After the reaction, the compound can be purified by a known method, such as Celite filtration, silica gel column chromatography, and organic solvent extraction.

The compound 2a can be obtained through the aforementioned procedure.

### [Method for Producing Pyrrole-Imidazole Polyamide]

The compound 3 obtained by the production method of the present invention can be used as a part of a pyrrole-imidazole polyamide.

The “pyrrole-imidazole polyamide” in the description herein is a straight-chain molecule containing an N-methylpyrrole unit (which may be hereinafter referred to as “Py”) and an N-methylimidazole unit (which may be hereinafter referred to as “Im”) bonded to each other.

The molecule preferably has a linker (such as γ-aminobutyric acid) intervening between the Py unit and/or the Im unit, so as to provide a macromolecule having a hairpin structure.

In the description herein, the pyrrole-imidazole polyamide may be in the form integrated with a drug (including a promoter and an inhibitor) for histone protein modification, such as a histone deacetylation inhibitor, or for DNA modification, such as a DNA methylation inhibitor.

It has been known that a pyrrole-imidazole polyamide can be incorporated into a DNA minor groove, in which Im/Py is bound to a G-C base pair, Py/Im is bound to a C-G base pair, and Py/Py is bound to an A-T base pair or a T-A base pair, and the interaction between G and Py is particularly strong, by which a pyrrole-imidazole polyamide is sequence-specifically bound to a DNA minor groove. The Im unit may be changed, for example, to a β-alanine (which may be hereinafter referred to as “Ala”) unit. One unit of an Ala unit may be contained per from 3 to 4 units of the Py unit or the Im unit.

Examples of the pyrrole-imidazole polyamide include

a compound represented by the general formula (4-1);

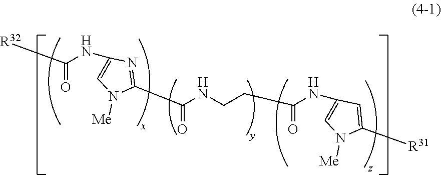

wherein x, y, and z each independently represent 0 or a natural number; and R³¹ and R³² each independently represent a protective group, and

a compound represented by the general formula (4-2);

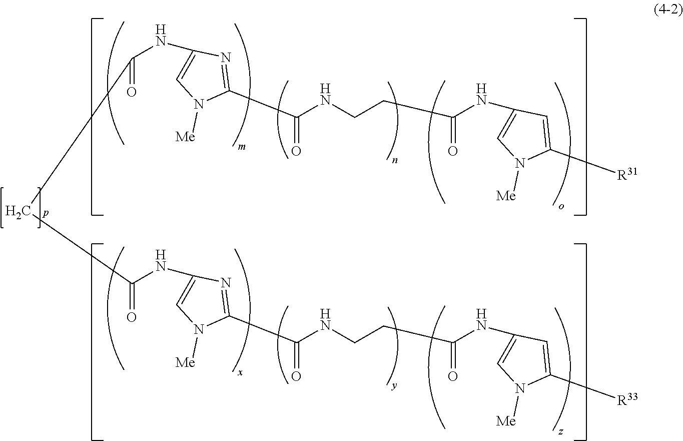

wherein m, n, o, p, x, y, and z each independently represent 0 or a natural number; and R³¹ and R³³ each independently represent a protective group.

Examples of the protective groups represented by R³¹ and R³³ include an (aminoalkyl)amino group, such as an aminopropylamino group and an N,N-dimethylaminopropylamino group, an acylamino group, such as an acetylamino group, and a carbamoyl group, such as an N-(aminopropyl)aminocarbonyl group and an N—(N′,N′-dimethylaminopropyl)aminocarbonyl group.

p may represent an integer of from 2 to 4, for example 3.

m+n+o and x+y+z each may be from 5 to 20, from 7 to 15, or from 7 to 10, corresponding to the length of the target DNA.

Examples of R³² include a methyl group and a tert-butoxy group.

In the compound, the Py unit, the Im unit, and the Ala unit may have any sequence, which is designed corresponding to the sequence of the target DNA.

The production method of the present invention is preferably used in the production of any structure of a Py-Py structure, an Im-Py structure, a Py-Im structure, and an Im-Im structure. The other units of the pyrrole-imidazole polyamide may be formed by known methods.

For the enhancement of the sequence specificity of the interaction between the pyrrole-imidazole polyamide and DNA, the target DNA sequence is preferably extended, and may be, for example, 4 or more base pairs, 5 or more base pairs, 6 or more base pairs, 7 or more base pairs, 8 or more base pairs, 9 or more base pairs, 10 or more base pairs, or any more.

The pyrrole-imidazole polyamide may be used, for example, for inhibiting the suppressed expression of a gene through methylation of DNA.

### [Novel Substance]

In the compound 2, a compound represented by the general formula (2aa):

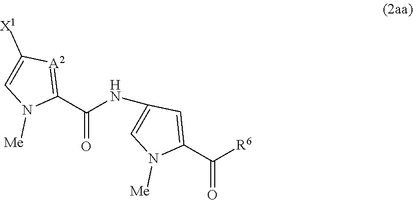

wherein A² represents N or CH; X¹ represents a halogen atom; and R⁶ represents an alkyloxy group having from 1 to 6 carbon atoms,

the general formula (2ab):

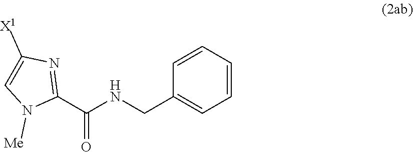

wherein X¹ represents a halogen atom,

the general formula (2ac):

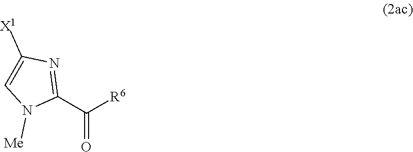

wherein X¹ represents a halogen atom; and R⁶ represents an alkyloxy group having from 1 to 6 carbon atoms, or

the general formula (2ad);

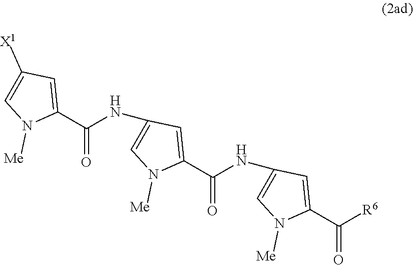

wherein X¹ represents a halogen atom; and R⁶ represents an alkyloxy group having from 1 to 6 carbon atoms, is a novel substance.

X¹ preferably represents an iodine atom, and R⁶ preferably represents an alkyloxy group having from 1 to 3 carbon atoms, and more preferably a methoxy group.

More specifically, examples thereof include the compound 2-3 and the compound 2-14 (which belong to the compound represented by the general formula (2aa)), the compound 2-10a and the compound 2-10b (which belong to the compound represented by the general formula (2ab)), the compound 2-5 and the compound 2-7 (which belong to the compound represented by the general formula (2ac)), and the compound 2-12 (which belongs to the compound represented by the general formula (2ad)), shown below.

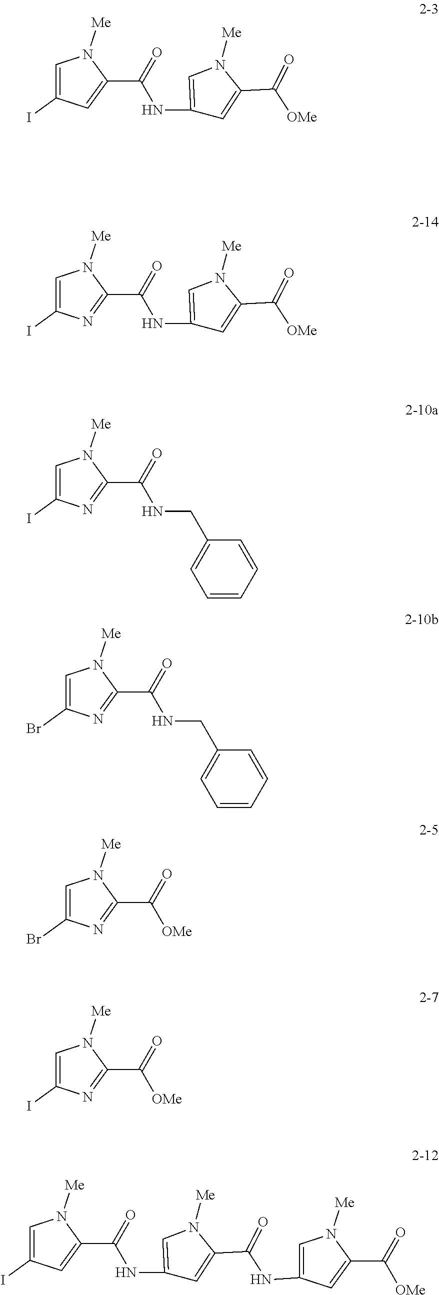

The compounds can be used as an intermediate for the synthesis of the pyrrole-imidazole polyamide. The compounds can be synthesized, for example, by the aforementioned halogenation step or the methods described in the examples later.

### EXAMPLES

The examples of the present invention shown below are only for exemplification, and do not restrict the technical scope of the present invention.

In the examples, the measurements were performed in the following manners.

### [Infrared Absorption Spectrum (IR)]

The infrared absorption (IR) spectrum was measured with a Fourier transformation infrared spectrometer equipped with ATR (attenuated total reflection).

### [Nuclear Magnetic Resonance (NMR)]

The nuclear magnetic resonance (NMR) spectrum was measured with a measuring equipment of 400 MHz. For the chemical shift in CDCl₃, tetramethylsilane (TMS) (0 ppm) was used as the internal standard in ¹H-NMR, and the signal of the solvent (CHCl₃ (77.0 ppm)) was used as the internal standard in ¹³C-NMR.

### [Mass Analysis]

The positive ion mass spectrum was measured by the electrospray ionization method (ESI-TOF).

### [Synthesis of Py-Py]

### Synthesis Example 1

### Synthesis Example 1-1: Synthesis of Compound 2-1

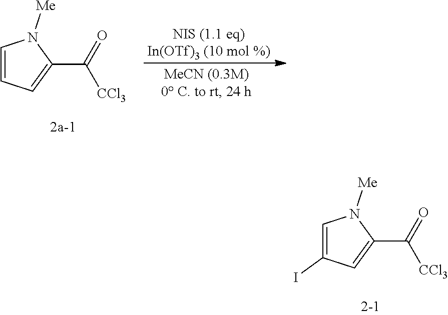

A solution of the compound 2a-1 (2.26 g, 10.0 mmol) in MeCN (33 mL) was stirred at 0° C., and In(OTf)₃ (562 mg, 1.0 mmol) and NIS (2.47 g, 11.0 mmol) were added thereto. After 24 hours, a saturated Na₂S₂O₃ aqueous solution was added to the reaction mixed liquid to terminate the reaction, and the reaction mixed liquid was extracted with ethyl acetate twice. The mixed organic phase was washed with a brine, dried over Na₂S₂O₃, filtered, and concentrated under reduced pressure.

The residue was purified by silica gel column chromatography (SiO₂, n-hexane/EtOAc=10/1), so as to provide the target compound 2-1 (3.46 g, 98% yield) as a white solid substance (melting point Mp.: 81-82° C.; Rf=0.36 (n-hexane/EtOAc=10/1). The compound 2-1 is a known compound (Journal of the American Chemical Society 1996, vol. 118, 6141).

### Data of Compound 2-1:

¹H NMR (400 MHz, CDCl₃) δ 3.97 (s, 3H), 7.01 (d, J=1.6 Hz, 1H), 7.56 (d, J=1.6 Hz, 1H);

¹³C NMR (100 MHz, CDCl₃) δ 38.6, 60.0, 95.7, 123.7, 129.9, 137.5, 172.1;

IR (ATR) ν 1678, 1458, 1411, 1359, 1193, 910, 850, 795, 753, 715, 686 cm⁻¹

### Synthesis Example 1-2: Synthesis of Compound 2-2

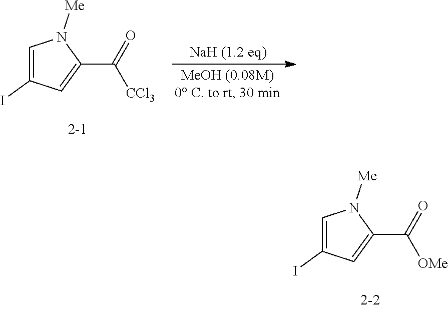

A solution of the compound 2-1 (335 mg, 0.95 mmol) in MeOH (10.6 mL) was stirred at 0° C., to which NaH (60% by mass oil dispersion, 45.6 mg, 1.14 mmol) was added. After stirring at room temperature for 30 minutes, a 1 N HCl aqueous solution was added to the reaction mixed liquid to terminate the reaction, and the reaction mixed liquid was concentrated under reduced pressure. The residue was diluted with ethyl acetate and water. The mixture was separated into two phases, and the aqueous phase was extracted with ethyl acetate twice. The mixed organic phase was washed with a brine, dried over Na₂SO₄, filtered, and concentrated under reduced pressure.

The residue was purified by silica gel column chromatography (SiO₂, n-hexane/EtOAc=10/1), so as to provide the target compound 2-2 (247 mg, 98% yield) as a white solid substance (melting point Mp.: 55-57° C.; Rf=0.31 (n-hexane/EtOAc=10/1).

### Data of Compound 2-2:

¹H NMR (400 MHz, CDCl₃) δ 3.81 (s, 3H), 3.91 (s, 3H), 6.82 (d, J=1.6 Hz, 1H), 7.01 (d, J=1.6 Hz, 1H);

¹³C NMR (100 MHz, CDCl₃) δ 36.8, 51.2, 58.7, 124.3, 133.6 (2C), 160.5;

IR (ATR) ν 3128, 2429, 1703, 1435, 1388, 1329, 1246, 1200, 1120 cm⁻¹

### Example 1

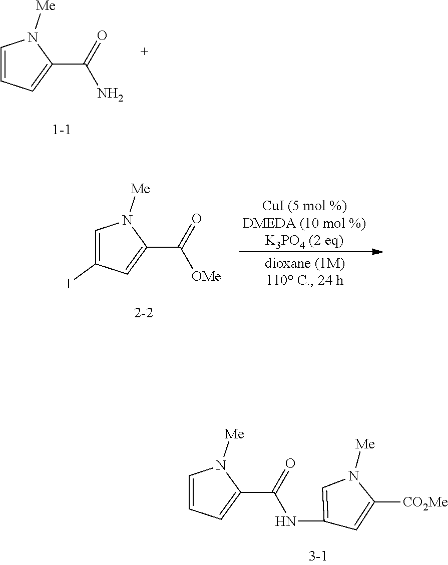 [SMILES: Cn1cccc1C(N)=O ; COC(=O)c1cc(I)cn1C ; COC(=O)c1cc(NC(=O)c2cccn2C)cn1C]

Under an argon atmosphere, the compound 2-2 (132 mg, 0.5 mmol), the compound 1-1 (68 mg, 0.55 mmol), CuI (4.8 mg, 0.025 mmol), and K₃PO₄ (212 mg, 1.0 mmol) were dissolved in dioxane (0.5 mL). N,N′-dimethylethylenediamine (which may be hereinafter referred simply to as “DMEDA”) (5.4 μL, 0.05 mmol) was added to the reaction product mixture, which was stirred at 110° C. for 24 hours. The resulting brown suspension was cooled to room temperature, filtered with Celite, and concentrated under reduced pressure.

The residue was purified by silica gel column chromatography (SiO₂, n-hexane/EtOAc=3/1), so as to provide the target compound 3-1 (123 mg, 94% yield) as a white solid substance (melting point: 110-112° C.; Rf=0.11 (n-hexane/EtOAc=3/1).

### Data of Compound 3-1:

¹H NMR (400 MHz, CDCl₃) δ 3.79 (s, 3H), 3.88 (s, 3H), 3.96 (s, 3H), 6.10 (dd, J=4.0, 2.4 Hz, 1H), 6.65 (dd, J=4.0, 1.6 Hz, 1H), 6.75 (d, J=2.4 Hz, 1H), 6.75 (dd, J=2.4, 1.6 Hz, 1H), 7.41 (d, J=2.4 Hz, 1H), 7.65 (s, 1H);

¹³C NMR (100 MHz, CDCl₃) δ 36.7, 36.7, 51.0, 107.3, 108.3, 111.8, 119.7, 120.9, 121.7, 125.3, 128.4, 159.2, 161.5;

IR (ATR) ν 1708, 1643, 1558, 1452, 1416, 1317, 1252, 1196, 1116, 736 cm⁻¹;

HRMS (ESI-TOF) m/z: [M+Na]⁺ Calcd. for C₁₃H₁₅N₃NaO₃⁺ 284.1006; Found 284.1014.

The compound 3-1 is a known compound (The Journal of Organic Chemistry 2003, vol. 68, 1158).

### Examples 2 to 4

The production methods of Examples 2 to 4 were performed in the same procedures as in Example 1 except that the compound 2 used for the reaction, the kind of the base, the solvent, and the concentration in the solvent were changed to those shown in Table 1 below.

The results of Examples are shown in Table 1.

TABLE 1

| 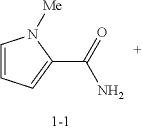 | | | | |
|---|---|---|---|---|
| 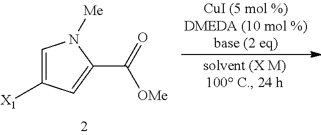 | | | | |
| 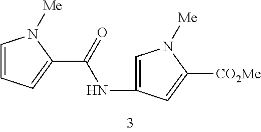 [SMILES: COC(=O)c1cc(NC(=O)c2cccn2C)cn1C] | | | | |
| Example | Base | X₁ | Solvent (conc.) | Result |
| 1 | K₃PO₄ | I | dioxane (1M) | 94% yield |
| 2 | Cs2CO₃ | I | dioxane (1M) | 92% yield |
| 3 | Cs2CO₃ | I | dioxane (0.5M) | 78% yield |
| 4 | Cs2CO₃ | I | toluene (1M) | 46% yield |

It was understood from the results that the target compounds were obtained with a high yield even in the case where Cs₂CO₃ was used as the base.

### [Synthesis of Py-Im]

### Synthesis Example 2: Synthesis of Compound 2-10a

### Synthesis Example 2-1: Synthesis of Compound 2-6

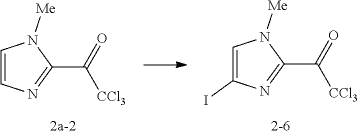

A solution of the compound 2a-2 (1.00 g, 4.42 mmol) and In(OTf)₃ (247.3 mg, 0.44 mmol) in MeCN (22.1 mL) was stirred at 0° C., to which DIH (1.80 g, 4.74 mmol) was added. The reaction was performed by gradually increasing the temperature to room temperature, and then continuously stirring at that temperature for 24 hours. A 5% Na₂S₂O₃ aqueous solution was added to the reaction mixed liquid to terminate the reaction, and the reaction mixed liquid was extracted with ethyl acetate. The mixed organic phase was washed with a brine, dried over Na₂S₂O₃, filtered, and concentrated under reduced pressure. The residue was purified by silica gel column chromatography (SiO₂, n-hexane/EtOAc=6/1), so as to provide the target compound 2-6 (700 mg, 45% yield) as a white solid substance.

### Data of Compound 2-6:

Melting point (Mp.) 112-113° C.;

¹H NMR (400 MHz, CDCl₃) δ 4.04 (s, 3H), 7.24 (s, 1H);

¹³C NMR (100 MHz, CDCl₃) δ 37.1, 83.8, 94.3, 134.1, 137.9, 171.2;

IR (ATR) ν 1691, 1407, 1367, 942, 852, 815, 741, 703, 612 cm⁻¹

### Synthesis Example 2-2: Synthesis of Compound 2-10a

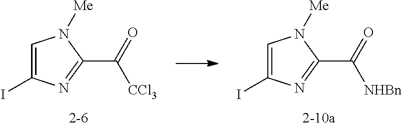

A solution of the compound 2-6 (140 mg, 0.397 mmol) in MeOH (2 mL) was stirred at 0° C., to which benzylamine (86 μL, 0.792 mmol) was added. The reaction was performed by gradually increasing the temperature to room temperature, and stirring at that temperature for 1 hour. Thereafter, the reaction mixed liquid was concentrated under reduced pressure. The residue was purified by silica gel column chromatography (SiO₂, n-hexane/EtOAc=4/1), so as to provide the target compound 2-10a (125 mg, 92% yield) as a white solid substance.

### Data of Compound 2-10:

Melting point (Mp.) 108-109° C.;

¹H NMR (400 MHz, CDCl₃) δ 4.06 (s, 3H), 4.54 (d, J=6.4 Hz, 2H), 7.05 (s, 1H), 7.26-7.37 (m, 5H), 7.64 (br-s, 1H);

¹³C NMR (100 MHz, CDCl₃) δ 35.7, 43.1, 80.6, 127.6, 127.9 (2C), 128.7 (2C), 130.8, 137.6, 140.8, 157.8;

IR (ATR) ν 1662, 1538, 1496, 1420, 1391, 1265, 1161, 947, 733, 698 cm⁻¹;

HRMS ((+)-ESI-TOF) m/z: [M+Na]⁺ Calcd. for C₁₂H₁₂IN₃NaO⁺ 363.9917; Found 363.9926.

### Synthesis Example 3: Synthesis of Compound 2-10b

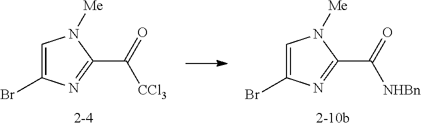

The compound 2-10b was prepared in the same manner as in Synthesis Example 2 except that the compound 2-4 was used instead of the compound 2-6.

### Data of Compound 2-10b:

Melting point (mp) 82-83° C.;

¹H NMR (400 MHz, CDCl₃) δ 4.06 (s, 3H), 4.53 (d, J=6.4 Hz, 2H), 6.93 (s, 1H), 7.26-7.36 (m, 5H), 7.59 (br-s, 1H);

¹³C NMR (100 MHz, CDCl₃) δ 35.7, 42.9, 113.7, 124.6, 127.4, 127.7 (2C), 128.5 (2C), 137.5, 138.4, 157.8;

IR (ATR) ν 1660, 1539, 1496, 1450, 1427, 1396, 1272, 957, 697, 625 cm⁻¹;

HRMS ((+)-ESI-TOF) m/z: [M+H]⁺ Calcd. for C₁₂H₁₃BrN₃O⁺ 294.0237; Found 294.0254.

### Example 5

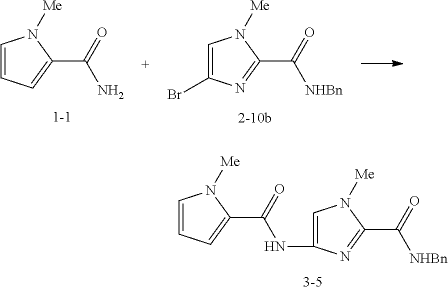

Under an argon atmosphere, the compound 2-10b (170.2 mg, 0.499 mmol), the compound 1-1 (68.5 mg, 0.552 mmol), CuI (9.3 mg, 0.049 mmol), and Cs₂CO₃ (326.4 mg, 1.00 mmol) were dissolved in dioxane (1.5 mL). N,N′-dimethylethylenediamine (23 μL, 0.205 mmol) was added to the reaction product mixture, which was stirred at 110° C. for 24 hours. The resulting brown suspension was cooled to room temperature, filtered with Celite, and concentrated under reduced pressure.

The residue was purified by silica gel column chromatography (SiO₂, n-hexane/EtOAc=1/2), so as to provide the target compound 3-5 (134.6 mg, 80% yield) as a pale yellow amorphous substance.

### Data of Compound 3-5:

¹H NMR (400 MHz, CDCl₃) δ 3.98 (s, 3H), 4.06 (s, 3H), 4.57 (d, J=6.0 Hz, 2H), 6.11-6.14 (m, 1H), 6.66-6.68 (m, 1H), 6.77-6.79 (m, 1H), 7.26-7.45 (m, 7H), 7.95 (s, 1H);

¹³C NMR (100 MHz, CDCl₃) δ 35.5, 36.8, 42.9, 107.6, 112.6, 113.5, 124.5, 127.4, 127.6 (2C), 128.6 (2C), 128.9, 133.7, 135.9, 137.9, 158.6, 158.7;

IR (ATR) ν 1652, 1521, 1470, 1409, 1361, 1246, 1116, 729, 696 cm⁻¹;

HRMS ((+)-ESI-TOF) m/z: [M+Na]⁺ Calcd. for C₁₈H₁₉N₅NaO₂⁺ 360.1431; Found. 360.1442.

### Examples 6 to 10

The production methods of Examples 6 to 10 were performed in the same procedures as in Example 5 except that the compound 2 used for the reaction, the amount of DMEDA added, the kind and the concentration of the base, and the concentration in the solvent were changed to those shown in Table 2 below.

The results of Examples are shown in Table 2.

TABLE 2

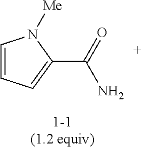

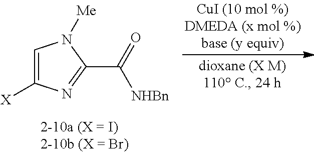

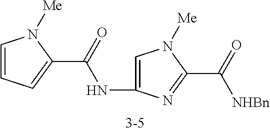

| Ex- | | | | | |
|---|---|---|---|---|---|
| am- | Sub- | DMEDA | | Concer- | |
| ple | strate | (x mol %) | Base (y equiv) | tation | Yield |
| 6 | 2-10a | 20 mol % | K₃PO₄ (2.0 equiv) | 0.33M | 63% yield |
| 7 | 2-10a | 20 mol % | Cs₂CO₃ (2.0 equiv) | 0.33M | 64% yield |
| 8 | 2-10a | 20 mol % | Cs₂CO₃ (2.0 equiv) | 0.5M | 75% yield |
| 9 | 2-10a | 40 mol % | Cs₂CO₃ (2.0 equiv) | 0.33M | 80% yield |
| 10 | 2-10b | 40 mol % | Cs₂CO₃ (2.0 equiv) | 0.33M | 62% yield |

### [Synthesis of Im-Py]

### Synthesis Example 4: Synthesis of Compound 1-3

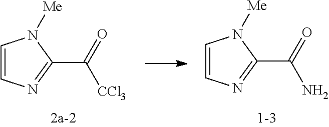

A solution of the compound 2a-2 (370.1 mg, 1.63 mmol) in MeOH (3.3 mL) was stirred at 0° C., to which a 25% NH₃ aqueous solution (3.3 mL) was added. After 15 minutes, EtOAc was added to the reaction mixture for diluting.

The organic phase was washed with a brine, dried over Na₂SO₄, filtered, and concentrated under reduced pressure. The residue was recrystallized from n-hexane and CHCl₃, so as to provide the target compound 1-3 (173.3 mg, 85% yield) as a white solid substance.

The compound 1-3 is a known compound (Bioorganic & Medicinal Chemistry Letters 2007, vol. 17, 6216).

### Example 11

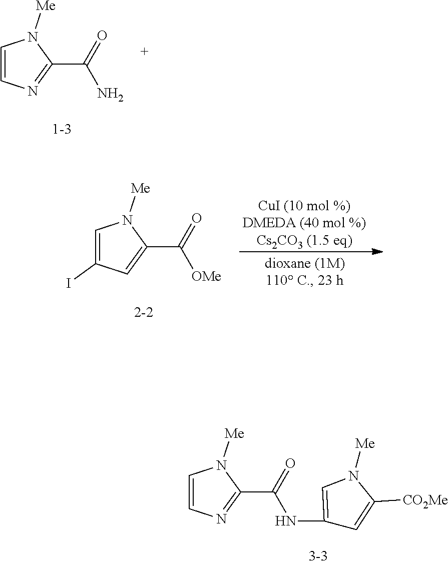 [SMILES: Cn1ccnc1C(N)=O ; COC(=O)c1cc(I)cn1C ; COC(=O)c1cc(NC(=O)c2nccn2C)cn1C]

Under an argon atmosphere, the compound 2-2 (132 mg, 0.5 mmol), the compound 1-3 (75 mg, 0.6 mmol), CuI (9.5 mg, 0.05 mmol), and Cs₂CO₃ (244 mg, 0.75 mmol) were dissolved in dioxane (0.5 mL). N,N′-dimethylethylenediamine (11 μL, 0.1 mmol) was added to the reaction product mixture, which was stirred at 110° C. for 23 hours. The resulting brown suspension was cooled to room temperature, filtered with Celite, and concentrated under reduced pressure.

The residue was purified by silica gel column chromatography (SiO₂, n-hexane/EtOAc=1/1), so as to provide the target compound 3-3 (89 mg, 68% yield) as a yellow solid substance (melting point mp: 124-125° C., Rf=0.25 (n-hexane/EtOAc=1/1)).

### Data of Compound 3-3:

¹H NMR (400 MHz, CDCl₃) δ 3.80 (s, 3H), 3.90 (s, 3H), 4.08 (s, 3H), 6.82 (d, J=1.6 Hz, 1H), 6.96 (s, 1H), 7.01 (s, 1H), 7.38 (d, J=1.6 Hz, 1H), 9.16 (s, 1H);

¹³C NMR (100 MHz, CDCl₃) δ 35.4, 36.6, 108.4, 120.1, 120.6, 121.4, 125.6, 127.7, 139.0, 156.3, 161.4;

IR (ATR) ν 1703, 1667, 1578, 1473, 1449, 1249, 1195, 1119, 1096, 777, 621 cm⁻¹

The compound 3-3 is a known compound (The Journal of Organic Chemistry. 2005, vol. 70, 10311).

### Examples 12 to 19

The compound 3-3 was obtained in the same procedures as in Example 11 except that DMEA, the solvent, the base, or the reaction time was changed to those shown in Table 3 below.

The results of Examples are shown in Table 3.

TABLE 3

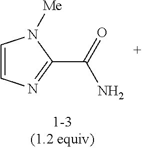

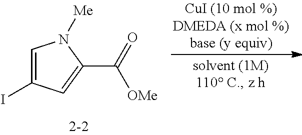 [SMILES: COC(=O)c1cc(I)cn1C]

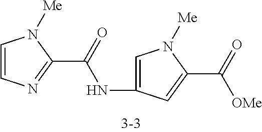 [SMILES: COC(=O)c1cc(NC(=O)c2nccn2C)cn1C]

| Ex- | DMEDA | | | Time | |
|---|---|---|---|---|---|
| ample | (x mol %) | Base (y equiv) | Solvent | (z h) | Yield |
| 12 | 40 mol % | Cs₂CO₃ (1.5 equiv) | dioxane | 23 h | 68% yield |
| 13 | 40 mol % | Cs₂CO₃ (1.5 equiv) | DMF | 23 h | 47% yield |
| 14 | 40 mol % | K₃PO₄ (1.5 equiv) | dioxane | 23 h | 38% yield |
| 15 | 20 mol % | K₃PO₄ (1.5 equiv) | dioxane | 24 h | 38% yield |
| 16 | 20 mol % | Cs₂CO₃ (1.5 equiv) | dioxane | 24 h | 68% yield |
| 17 | 40 mol % | Cs₂CO₃ (1.5 equiv) | dioxane | 24 h | 74% yield |
| 18 | 40 mol % | K₃PO₄ (2 equiv) | dioxane | 24 h | 55% yield |
| 19 | 40 mol % | Cs₂CO₃ (2 equiv) | dioxane | 24 h | 63% yield |

### [Synthesis of Im-Im]

### Synthesis Example 5: Synthesis of Compound 2-5

### Synthesis Example 5-1: Synthesis of Compound 2-4

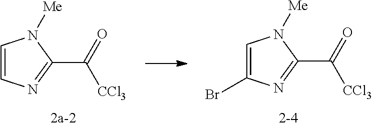

A solution of the compound 2a-2 (3.41 g, 15 mmol) in MeCN (30 mL) was stirred at 0° C., to which DBH (2.58 g, 9.0 mmol) was added. The reaction was performed by gradually increasing the temperature to room temperature, and then continuously stirring at that temperature for 24 hours. A 5% Na₂S₂O₃ aqueous solution was added to the reaction mixed liquid to terminate the reaction, and the reaction mixed liquid was extracted with ethyl acetate. The mixed organic phase was washed with a brine, dried over Na₂S₂O₃, filtered, and concentrated under reduced pressure. The residue was recrystallized from acetone, so as to provide the target compound 2-4 (4.17 g, 91% yield) as a white solid substance.

### Data of Compound 2-4:

Melting point (Mp.) 117-118° C.;

¹H NMR (400 MHz, CDCl₃) δ 4.04 (s, 3H), 7.16 (s, 1H);

¹³C NMR (100 MHz, CDCl₃) δ 37.3, 94.2, 116.4, 128.3, 135.5, 171.3;

IR (ATR) ν 1694, 1413, 1377, 954, 818, 750, 721 cm⁻¹

### Synthesis Example 5-2: Synthesis of Compound 2-5

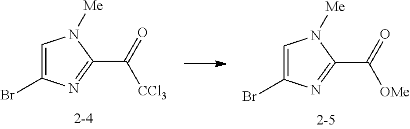

A solution of the compound 2-4 (1.91 g, 6.24 mmol) and DMAP (305 mg, 2.50 mmol) in MeOH (31 mL) was stirred at room temperature for 30 minutes. The reaction mixed liquid was concentrated under reduced pressure, and the resulting residue was purified by silica gel column chromatography (SiO₂, EtOAc), so as to provide the target compound 2-5 (1.32 g, 96% yield) as a white solid substance.

### Data of Compound 2-5:

Melting point (Mp.) 94-95° C.;

¹H NMR (400 MHz, CDCl₃) δ 3.94 (s, 3H), 4.01 (s, 3H), 7.03 (s, 1H);

¹³C NMR (100 MHz, CDCl₃) δ 36.0, 52.4, 115.6, 125.7, 135.9, 158.6;

IR (ATR) ν 1713, 1446, 1243, 1122, 953 cm⁻¹;

HRMS ((+)-ESI-TOF) m/z: [M+H]⁺ Calcd. for C₆H₈BrN₂O₂⁺ 218.9764; Found 218.9749.

### Synthesis Example 6: Synthesis of Compound 2-7

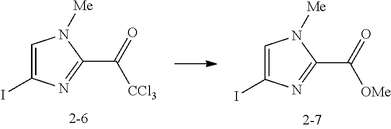

The compound 2-7 was obtained from the compound 2-6 in the same procedure as in Synthesis Example 5-2 (92% yield, white solid substance).

### Data of Compound 2-7:

Melting point (Mp.) 140-141° C.;

¹H NMR (400 MHz, CDCl₃) δ 3.94 (s, 3H), 4.01 (s, 3H), 7.14 (s, 1H);

¹³C NMR (100 MHz, CDCl₃) δ 35.8, 52.3, 82.3, 131.8, 138.1, 158.2;

IR (ATR) ν 1707, 1446, 1270, 1232, 1134, 943 cm⁻¹;

HRMS ((+)-ESI-TOF) m/z: [M+H]⁺ Calcd. for C₆H₈IN₂O₂⁺ 266.9625; Found 266.9614.

### Example 20

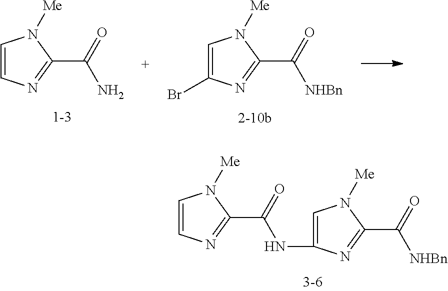

Under an argon atmosphere, the compound 2-10b (170.4 mg, 0.50 mmol), the compound 1-3 (75.3 mg, 0.60 mmol), CuI (9.1 mg, 0.048 mmol), and Cs₂CO₃ (245.1 mg, 0.75 mmol) were dissolved in dioxane (1.5 mL). N,N′-dimethylethylenediamine (23 μL, 0.205 mmol) was added to the reaction product mixture, which was stirred at 110° C. for 24 hours. The resulting dark green suspension was cooled to room temperature, filtered with Celite, and concentrated under reduced pressure.

The residue was purified by silica gel column chromatography (SiO₂, n-hexane/EtOAc=1/2), so as to provide the target compound 3-6 (91.6 mg, 54% yield) as a pale yellow amorphous substance.

### Data of Compound 3-6:

Melting point (Mp.) 148-149° C.;

¹H NMR (400 MHz, CDCl₃) δ 4.05 (s, 3H), 4.06 (s, 3H), 4.55 (d, J=6.4 Hz, 2H), 6.97 (s, 1H), 7.02 (s, 1H), 7.25-7.36 (m, 5H), 7.42 (s, 1H), 7.65 (t, J=6.4 Hz, 1H), 9.54 (s, 1H);

¹³C NMR (100 MHz, CDCl₃) δ 35.6, 35.6, 42.9, 113.7, 126.0, 127.4, 127.6 (2C), 128.1, 128.6 (2C), 134.2, 135.3, 138.0, 138.3, 156.2, 158.8;

IR (ATR) ν 1663, 1526, 1470, 1284, 889, 730, 698 cm⁻¹;

HRMS ((+)-ESI-TOF) m/z: [M+H]⁺ Calcd. for C₁₇H₁₉N₆O₂⁺ 339.1564; Found 339.1554.

### Examples 21 to 25

The production methods of Examples 21 to 25 were performed in the same procedures as in Example 20 except that the compound 2 used for the reaction, the amount of DMEDA added, the kind and the concentration of the base, and the concentration in the solvent were changed to those shown in Table 4 below.

The results of Examples are shown in Table 4.

TABLE 4

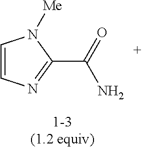

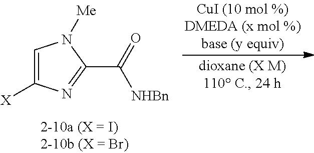

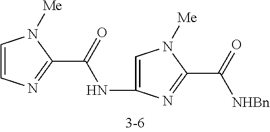

| Ex- | | | | | |
|---|---|---|---|---|---|
| am- | Sub- | DMEDA | | Concer- | |
| ple | strate | (x mol %) | Base (y equiv) | tation | Yield |
| 21 | 2-10a | 40 mol % | Cs₂CO₃ (1.5 equiv) | 0.33M | 54% yield |
| 22 | 2-10a | 40 mol % | Cs₂CO₃ (2.0 equiv) | 0.33M | 41% yield |
| 23 | 2-10a | 40 mol % | K₃PO₄ (1.5 equiv) | 0.33M | 51% yield |
| 24 | 2-10a | 40 mol % | Cs₂CO₃ (1.5 equiv) | 0.5M | 44% yield |
| 25 | 2-10b | 40 mol % | Cs₂CO₃ (1.5 equiv) | 0.33M | 6% yield |

### [Synthesis of Py-Py-Py]

### Synthesis Example 7: Synthesis of I-Py-Py (Step 1a)

### Synthesis Example 7-1: Synthesis of Compound 2a-1-1

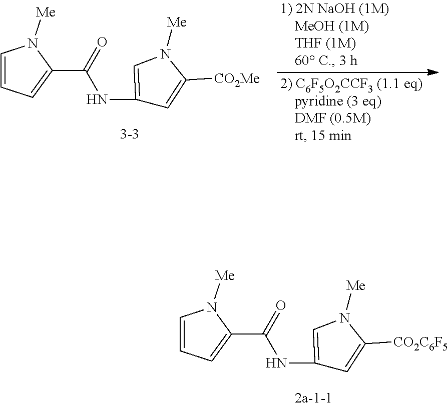

A solution of the compound 3-1 (174.5 mg, 0.67 mmol) in MeOH (0.67 mL) and THF (0.67 mL) was stirred at room temperature, to which a 2 N NaOH aqueous solution (0.67 mL) was added. After stirring at 60° C. for 3 hours, a 1 N HCl aqueous solution was added to the reaction mixture to terminate the reaction, and the reaction mixture was extracted with ethyl acetate twice. The mixed organic phase was washed with a brine, dried over Na₂S₂O₃, filtered, and concentrated under reduced pressure. The resulting residue was used for the subsequent reaction without purification. A solution of the resulting product in pyridine (0.16 mL) and DMF (1.2 mL) was stirred at room temperature, to which C₆F₅O₂CCF₃ (0.125 mL, 0.73 mmol) was added, and the resulting mixture was stirred at room temperature for 15 minutes. A 1 N HCl aqueous solution was added to the reaction mixture to terminate the reaction, and the reaction mixture was extracted with ethyl acetate twice. The mixed organic phase was washed with a NaHCO₃ aqueous solution and a brine, dried over Na₂S₂O₃, filtered, and concentrated under reduced pressure.

The residue was purified by silica gel column chromatography (SiO₂, n-hexane/EtOAc=4/1), so as to provide the target compound 2a-1-1 (265 mg, 96% yield) as a white solid substance (melting point Mp.: 149-152° C., Rf=0.38 (n-hexane/EtOAc=2/1)).

Data of Compound 2a-1-1;

¹H NMR (400 MHz, CDCl₃) δ 3.92 (s, 3H), 3.98 (s, 3H), 6.11-6.14 (m, 1H), 6.66-6.67 (m, 1H), 6.78 (br-s, 1H), 7.05-7.07 (m, 1H), 7.60-7.62 (m, 2H);

¹³C NMR (100 MHz, CDCl₃) δ 36.7 (2C), 107.6, 111.0, 112.0, 116.6, 122.9, 123.9, 125.3, 128.7, 136.7-136.9 (m), 139.1-139.3 (m), 140.5-140.7 (m), 142.9-143.1 (m), 156.2, 159.4;

IR (ATR) ν 1742, 1644, 1522, 1412, 1316, 1231, 1192, 1109, 1038, 738 cm⁻¹;

HRMS (ESI-TOF) m/z; [M+Na]⁺ Calcd. for C₁₈H₁₂F₅N₃NaO₃⁺ 436.0691; Found 436.0687.

### Synthesis Example 7-2 (Step 1a-1): Synthesis of Compound 2a-2-1

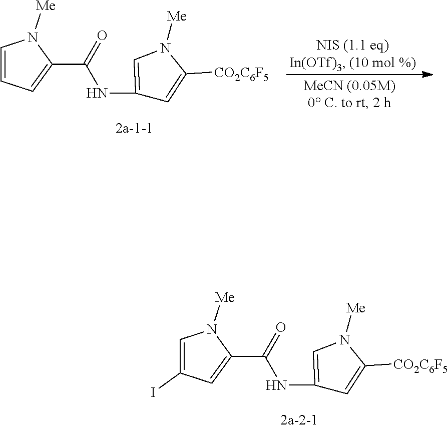

A solution of the compound 2a-1-1 (82.7 mg, 0.2 mmol) in MeCN (2 mL) was stirred at 0° C., to which In(OTf)₃ (11.2 mg, 0.02 mmol) and NIS (50 mg, 0.22 mmol) were added. After stirring at room temperature for 2 hours, a saturated Na₂S₂O₃ aqueous solution was added to the reaction mixed liquid to terminate the reaction, and the reaction mixed liquid was extracted with ethyl acetate twice. The mixed organic phase was washed with a brine, dried over Na₂S₂O₃, filtered, and concentrated under reduced pressure.

The residue was purified by silica gel column chromatography (SiO₂, n-hexane/EtOAc=5/1), so as to provide the target compound 2a-2-1 (92.9 mg, 86% yield) as a white solid substance (Mp.: 186-188° C.; Rf=0.41 (n-hexane/EtOAc=2/1).

Data of Compound 2a-2-1:

¹H NMR (400 MHz, CDCl₃) δ 3.93 (s, 3H), 3.96 (s, 3H), 6.73 (d, J=2.0 Hz, 1H), 6.83 (d, J=2.0 Hz, 1H), 7.06 (d, J=2.0 Hz, 1H), 7.55 (br-s, 1H), 7.58 (d, J=2.0 Hz, 1H);

¹³C NMR (100 MHz, CDCl₃) δ 36.7, 36.8, 58.3, 111.0, 16.7, 118.8, 122.5, 123.9, 127.4, 133.1, 136.6-136.9 (m), 139.0-139.4 (m), 140.4-140.7 (m), 142.9-143.1 (m), 156.2, 158.0;

IR (ATR) ν 1717, 1652, 1559, 1519, 1396, 1040, 993, 808 cm⁻¹;

HRMS (ESI-TOF) m/z: [M+Na]⁺ Calcd. for C₁₈H₁₁F₅IN₃NaO₃⁺ 561.9657; Found 561.9664.

### Synthesis Example 7-3 (Step 1a-2): Synthesis of Compound 2-3

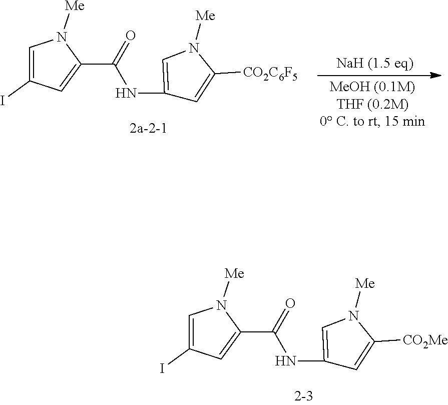

A solution of the compound 2a-2-1 (27 mg, 0.05 mmol) in MeOH (0.5 mL) and THF (0.25 mL) was stirred at 0° C., to which NaH (60% by mass oil dispersion, 3 mg, 0.075 mmol) was added. After stirring at room temperature for 15 minutes, a saturated NH₄Cl aqueous solution was added to the reaction mixed liquid to terminate the reaction, and the reaction mixed liquid was concentrated under reduced pressure. The residue was diluted with ethyl acetate and water. The mixture was separated into two phases, and the aqueous phase was extracted with ethyl acetate twice. The mixed organic phase was washed with a brine, dried over Na₂SO₄, filtered, and concentrated under reduced pressure.

The residue was purified by silica gel column chromatography (SiO₂, n-hexane/EtOAc=3.5/1), so as to provide the target compound 2-3 (18.7 mg, 97% yield) as a white solid substance.

### Data of Compound 2-3:

¹H NMR (400 MHz, CDCl₃) δ 3.81 (s, 3H), 3.88 (s, 3H), 3.94 (s, 3H), 6.72 (d, J=1.6 Hz, 1H), 6.74 (d, J=1.6 Hz, 1H), 6.79 (d, J=1.6 Hz, 1H), 7.38 (d, J=1.6 Hz, 1H), 7.63 (br-s, 1H);

¹³C NMR (100 MHz, CDCl₃) δ 36.8, 36.8, 51.1, 58.1, 108.3, 118.5, 119.8, 120.9, 121.3, 127.5, 132.6, 157.8, 161.4;

IR (ATR) ν 1706, 1645, 1559, 1450, 1399, 1322, 1248, 1206, 1111 cm⁻¹;

HRMS (ESI-TOF) m/z: [M+Na]⁺ Calcd. for C₁₃H₁₄IN₃NaO₃⁺ 409.9972; Found 409.9958.

### Synthesis Example 8: Synthesis of I-Py-Py (Step 1b)

### Synthesis Example 8-1: Synthesis of Compound 2a-3-1

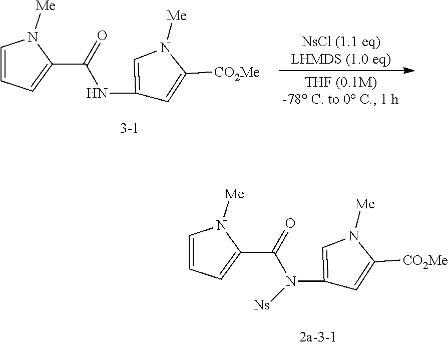

A solution of the compound 3-1 (78.4 mg, 0.3 mmol) in THF (3.0 mL) was stirred at −78° C., to which LHMDS (0.3 mL, 1.0 M in THF, 0.3 mmol) was added. After stirring at −78° C. for 15 minutes, NsCl (73.1 mg, 0.33 mmol) was added to the reaction mixture at 0° C. After stirring at 0° C. for 1 hour, a saturated NH₄Cl aqueous solution was added to the reaction mixed liquid to terminate the reaction, and the reaction mixed liquid was extracted with ethyl acetate twice. The mixed organic phase was washed with a brine, dried over Na₂S₂O₃, filtered, and concentrated under reduced pressure.

The residue was purified by silica gel column chromatography (SiO₂, n-hexane/EtOAc=2/1), so as to provide the target compound 2a-3-1 (106 mg, 79% yield) as a pale yellow amorphous substance (Rf=0.08 (n-hexane/EtOAc=2/1).

Data of Compound 2a-3-1;

¹H NMR (400 MHz, CDCl₃) δ 3.80 (s, 3H), 3.81 (s, 3H), 3.94 (s, 3H), 5.96 (dd, J=4.4, 2.4 Hz, 1H), 6.37 (dd, J=4.4, 1.6 Hz, 1H), 6.73 (dd, J=2.4, 1.6 Hz, 1H), 7.01 (d, J=1.6 Hz, 1H), 7.04 (d, J=1.6 Hz, 1H), 7.69-7.77 (m, 3H), 8.44-8.47 (m, 1H);

¹³C NMR (100 MHz, CDCl₃) δ 37.3, 37.4, 51.2, 108.2, 117.6, 117.9, 120.8, 121.9, 123.1, 124.1, 129.1, 130.9, 131.6, 132.7, 134.1, 134.2, 148.2, 161.1, 161.2;

IR (ATR) ν 1714, 1544, 1443, 1404, 1372, 1266, 1176, 1105, 739, 655 cm⁻¹;

HRMS (ESI-TOF) m/z: [M+Na]⁺ Calcd. for C₁₉H₁₈N₄NaO₇S⁺ 469.0788; Found 469.0783.

### Synthesis Example 8-2 (Step 1b-1); Synthesis of Compound 2a-4-1

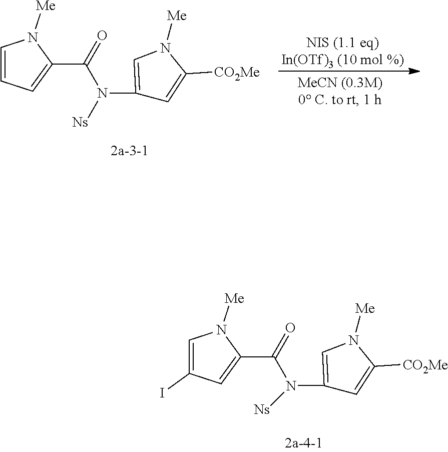

A solution of the compound 2a-3-1 (670 mg, 1.5 mmol) in MeCN (15 mL) was stirred at 0° C., to which In(OTf)₃ (84 mg, 0.15 mmol) and NIS (371 mg, 1.65 mmol) were added. After stirring at room temperature for 1 hour, a saturated Na₂S₂O₃ aqueous solution was added to the reaction mixed liquid to terminate the reaction, and the reaction mixed liquid was extracted with ethyl acetate twice. The mixed organic phase was washed with a brine, dried over Na₂S₂O₃, filtered, and concentrated under reduced pressure.

The residue was purified by silica gel column chromatography (SiO₂, n-hexane/EtOAc=2.5/1), so as to provide the target compound 2a-4-1 (781 mg, 91% yield) as a yellow amorphous substance (Rf=0.11 (n-hexane/EtOAc=2/1).

Data of Compound 2a-4-1;

¹H NMR (400 MHz, CDCl₃) δ 3.80 (s, 3H), 3.82 (s, 3H), 3.96 (s, 3H), 6.43 (d, J=2.0 Hz, 1H), 6.76 (d, J=2.0 Hz, 1H), 6.98 (d, J=2.0 Hz, 1H), 7.03 (d, J=2.0 Hz, 1H), 7.71-7.77 (m, 3H), 8.41 (dd, J=6.0, 3.2 Hz, 1H);

¹³C NMR (100 MHz, CDCl₃) δ 37.3, 37.5, 51.3, 59.1, 116.9, 117.8, 122.1, 124.2, 125.3, 126.4, 129.1, 131.7, 132.3, 134.3, 134.5, 134.9, 148.2, 159.9, 161.1;

IR (ATR) ν 1714, 1543, 1442, 1367, 1267, 1175, 1106, 1054, 739 cm⁻¹;

HRMS (ESI-TOF) m/z: [M+Na]⁺ Calcd. for C₁₉H₁₇IN₄NaO₆S₂⁺ 594.0755; Found 594.9771.

### Synthesis Example 8-3 (Step 1b-2): Synthesis of Compound 2-3

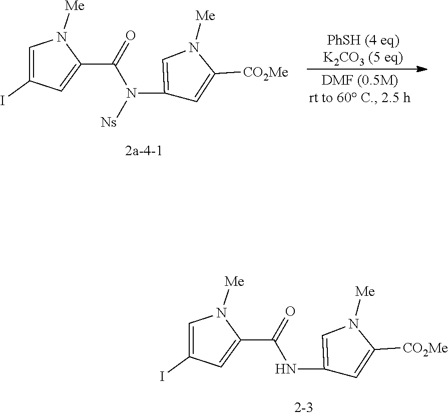

A solution of the compound 2a-4-1 (114 mg, 0.2 mmol) and K₂CO₃ (138 mg, 1.0 mmol) in DMF (0.4 mL) was stirred at room temperature, to which thiophenol (0.08 mL, 0.8 mmol) was added. After stirring at 60° C. for 2.5 hours, a saturated NaHCO₃ aqueous solution was added to the reaction mixed liquid to terminate the reaction, and the reaction mixed liquid was extracted with ethyl acetate twice. The mixed organic phase was washed with a brine, dried over Na₂S₂O₃, filtered, and concentrated under reduced pressure.

The residue was purified by silica gel column chromatography (SiO₂, n-hexane/EtOAc=2/1), so as to provide the target compound 2-3 (32 mg, 41% yield) as a white solid substance (Mp.: 155-157° C., Rf=0.25 (n-hexane/EtOAc=2/1).

### Comparative Synthesis Example 1: Synthesis Example of I-Py-Py (3)

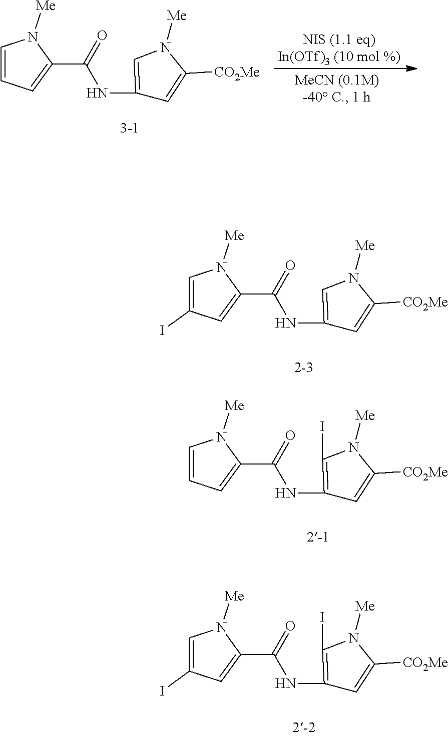 [SMILES: COC(=O)c1cc(NC(=O)c2cccn2C)cn1C ; COC(=O)c1cc(NC(=O)c2cc(I)cn2C)cn1C ; COC(=O)c1cc(NC(=O)c2cccn2C)c(I)n1C ; COC(=O)c1cc(NC(=O)c2cc(I)cn2C)c(I)n1C]

A solution of the compound 3-1 (26.1 mg, 0.10 mmol) and In(OTf)₃ (5.6 mg, 0.01 mmol) in MeCN (1 mL) was stirred at −40° C., to which NIS (24.7 mg, 0.11 mmol) was added. After 1 hour, a saturated Na₂S₂O₃ aqueous solution was added to the reaction mixed liquid to terminate the reaction, and the reaction mixed liquid was extracted with ethyl acetate twice. The mixed organic phase was washed with a brine, dried over Na₂S₂O₃, filtered, and concentrated under reduced pressure.

The residue was purified by silica gel column chromatography (SiO₂, n-hexane/EtOAc=2/1), so as to provide the target compound 2-3 (10.7 mg, 31% yield) as a white solid substance. The formation of the compound 2′-1 and the compound 2′-2 in the mixed liquid after completing the reaction was confirmed by TLC.

### Synthesis Example 9: Synthesis of BocHN-Py-CONH₂

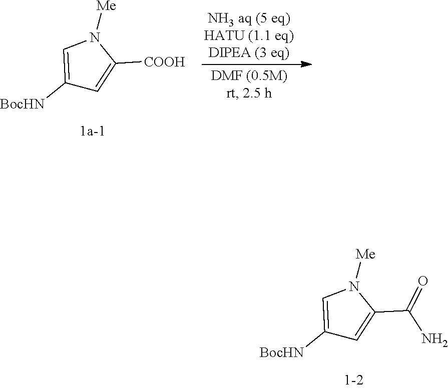 [SMILES: Cn1cc(NC(=O)OC(C)(C)C)cc1C(=O)O ; Cn1cc(NC(=O)OC(C)(C)C)cc1C(N)=O]

A solution of the compound 1a-1 (1.27 g, 5.3 mmol) and HATU (2.2 g, 5.8 mmol) in DMF (10.6 mL) was stirred at room temperature, to which N,N-diisopropylethylamine (DIPEA) (2.7 mL, 15.9 mmol) and an ammonia aqueous solution (25%, 2.0 mL) were added. After stirring for 2.5 hours, a 1 N KHSO₄ aqueous solution was added to the reaction mixed liquid to terminate the reaction, and the reaction mixed liquid was extracted with ethyl acetate twice. The mixed organic phase was washed with a NaHCO₃ saturated aqueous solution and a brine, dried over Na₂SO₄, filtered, and concentrated under reduced pressure.

The residue was purified by silica gel column chromatography (SiO₂, n-hexane/EtOAc=1/1.5), so as to provide the target compound 1-2 (836 mg, 70% yield) as a yellow amorphous substance (Rf=0.08 (n-hexane/EtOAc=1/1.5)).

### Data of Compound 1-2:

¹H NMR (400 MHz, CDCl₃) δ 1.49 (s, 9H), 3.87 (s, 3H), 5.77 (br-s, H), 6.52 (br-s, 1H), 6.54 (br-s, 1H), 6.87 (br-s, 1H);

¹³C NMR (100 MHz, CDCl₃) δ 28.3 (3C), 36.6, 80.1, 104.5, 118.5, 121.8, 122.2, 153.3, 163.5;

IR (ATR) ν 3332, 1699, 1653, 1580, 1456, 1392, 1251, 1164, 998, 777 cm⁻¹;

HRMS (ESI-TOF) m/z: [M+Na]⁺ Calcd. for C₁₁H₁₇N₃NaO⁺ 262.1162; Found 262.1164.

The compound 1a-1 is a known compound (The Journal of Organic Chemistry 2004, vol. 69, 8151).

### Example 26

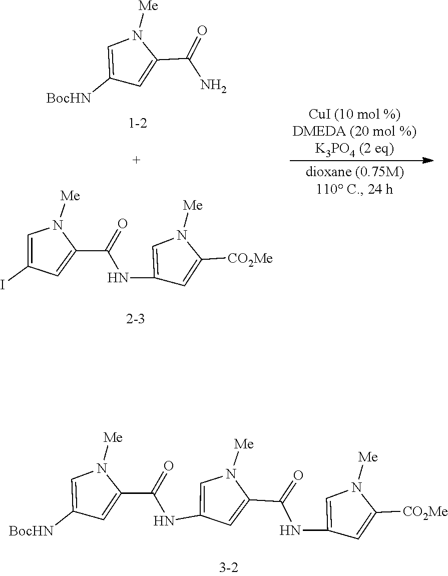 [SMILES: Cn1cc(NC(=O)OC(C)(C)C)cc1C(N)=O ; COC(=O)c1cc(NC(=O)c2cc(I)cn2C)cn1C ; COC(=O)c1cc(NC(=O)c2cc(NC(=O)c3cc(NC(=O)OC(C)(C)C)cn3C)cn2C)cn1C]

Under an argon atmosphere, the compound 1-2 (77.4 mg, 0.2 mmol), the compound 2-3 (77.4 mg, 0.2 mmol), CuI (3.8 mg, 0.02 mmol), and K₃PO₄ (85 mg, 0.4 mmol) were dissolved in dioxane (0.27 mL). N,N′-dimethylethylenediamine (DMEDA) (4.3 μL, 0.04 mmol) was added to the reaction product mixture, which was stirred at 110° C. for 24 hours. The resulting brown suspension was cooled to room temperature, filtered with Celite, and concentrated under reduced pressure.

The residue was purified by silica gel column chromatography (SiO₂, n-hexane/EtOAc=1/1.5), so as to provide the target compound 3-2 (70.2 mg, 70% yield) as a pale yellow amorphous substance.

The compound 3-2 is a known compound (Journal of the American Chemical Society. 2000, vol. 122, 6382).

### Example 27

Under an argon atmosphere, the compound 1-2 (77.4 mg, 0.2 mmol), the compound 2-3 (77.4 mg, 0.2 mmol), CuI (3.8 mg, 0.02 mmol), and Cs₂CO₃ (130 mg, 0.4 mmol) were dissolved in dioxane (0.27 mL). N,N′-dimethylethylenediamine (4.3 μL, 0.04 mmol) was added to the reaction product mixture, which was stirred at 110° C. for 24 hours. The resulting brown suspension was cooled to room temperature, filtered with Celite, and concentrated under reduced pressure.

The residue was purified by silica gel column chromatography (SiO₂, n-hexane/EtOAc=1/1.5), so as to provide the target compound 3-2 (62.2 mg, 62% yield) as a yellow amorphous substance.

### [Synthesis of Py-Im-Py]

### Synthesis Example 10: Synthesis of Compound 2-14

### Synthesis Example 10-1: Synthesis of Compound 6

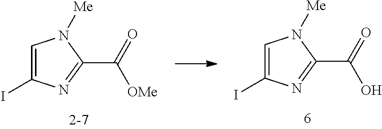 [SMILES: COC(=O)c1nc(I)cn1C ; Cn1cc(I)nc1C(=O)O]

A solution of the compound 2-7 (316.3 mg, 1.19 mmol) in MeOH (2.4 mL) and THF (2.4 mL) was stirred, to which a 2 N NaOH aqueous solution (1.2 mL) was added. The reaction mixture was heated to 60° C. for 2 hours. Thereafter, the reaction mixture was cooled to room temperature, and the mixture was extracted with Et₂O. The aqueous phase was acidified with a 1 N hydrochloric acid aqueous solution, and the half amount of water was distilled off under reduced pressure. A white solid substance was collected by filtration, so as to provide the target compound 6 (113.7 mg, 38% yield).

### Synthesis Example 10-2: Synthesis of Compound 2-14

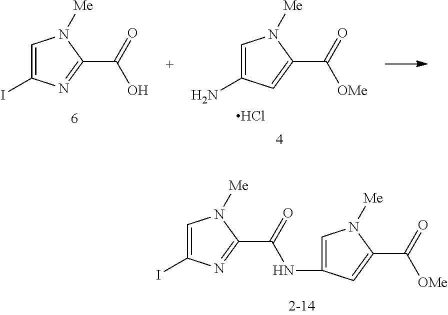 [SMILES: Cn1cc(I)nc1C(=O)O ; COC(=O)c1cc(N)cn1C ; Cl ; COC(=O)c1cc(NC(=O)c2nc(I)cn2C)cn1C]

A solution of the compound 6 (113.7 mg, 0.451 mmol) and HATU (224.7 mg, 0.591 mmol) in DMF (1 mL) was stirred at 0° C., to which N,N-diisopropylethylamine (DIPEA) (0.27 mL, 1.59 mmol) and the compound 4 (90.0 mg, 0.472 mmol) were added. The resulting solution was stirred at room temperature for 20 hours. A 1 N KHSO₄ aqueous solution was added to the reaction mixed liquid to terminate the reaction, and the reaction mixed liquid was extracted with ethyl acetate twice. The mixed organic phase was washed with water and a brine, dried over Na₂SO₄, and concentrated under reduced pressure, and the resulting residue was purified by silica gel column chromatography (SiO₂, n-hexane/EtOAc=2/1), so as to provide the target compound 2-14 (91.6 mg, 52% yield) as a white solid substance.

### Data of Compound 2-14:

¹H NMR (400 MHz, CDCl₃) δ 3.82 (s, 3H), 3.91 (s, 3H), 4.09 (s, 3H), 6.82 (d, J=2.0 Hz, 1H), 7.08 (s, 1H), 7.39 (d, J=2.0 Hz, 1H), 8.93 (s, 1H);

¹³C NMR (100 MHz, CDCl₃) δ 35.7, 36.8, 51.1, 80.6, 108.2, 120.0, 120.5, 120.8, 131.1, 140.7, 154.8, 161.3

### Example 28

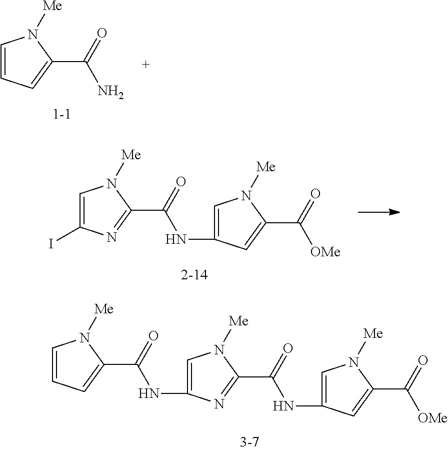 [SMILES: Cn1cccc1C(N)=O ; COC(=O)c1cc(NC(=O)c2nc(I)cn2C)cn1C ; COC(=O)c1cc(NC(=O)c2nc(NC(=O)c3cccn3C)cn2C)cn1C]

Under an argon atmosphere, the compound 2-14 (77.6 mg, 0.2 mmol), the compound 1-1 (27.3 mg, 0.22 mmol), CuI (3.8 mg, 0.02 mmol), and Cs₂CO₃ (130.3 mg, 0.4 mmol) were dissolved in dioxane (1 mL). N,N′-dimethylethylenediamine (DMEDA) (8.6 mL, 0.08 mmol) was added to the reaction product mixture, which was stirred at 110° C. for 24 hours. The resulting suspension was cooled to room temperature, filtered with Celite, and concentrated under reduced pressure.

The residue was purified by silica gel column chromatography (SiO₂, n-hexane/EtOAc=2/1), so as to provide the target compound 3-7 (18.5 mg, 24% yield) as a pale yellow amorphous substance.

### Data of Compound 3-7:

¹H NMR (400 MHz, CDCl₃) δ 3.82 (s, 3H), 3.92 (s, 3H), 4.00 (s, 3H), 4.08 (s, 3H), 6.16 (dd, J=2.4 Hz, 4.0 Hz, 1H), 6.72-6.80 (m, 3H), 7.43 (d, J=2.0 Hz, 1H), 7.48 (s, 1H), 8.01 (s, 1H), 8.78 (s, 1H);

HRMS ((+)-ESI-TOF) m/z: [M+Na]⁺ Calcd. for C₁₈H₂₀N₆NaO₄⁺ 407.1438; Found 407.1450.

### [Synthesis of Py-Py-Py-Py]

### Synthesis Example 11: Synthesis of Py-Py-CONH₂

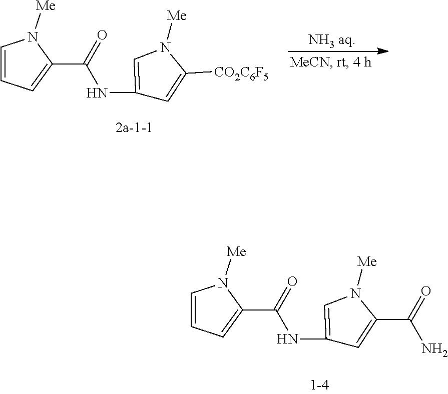

A solution of the compound 2a-1-1 (605 mg, 1.46 mmol) in MeCN (30 mL) was stirred at room temperature, to which an NH₃ aqueous solution (2.2 mL, 29.2 mmol) was added, and the resulting mixture was stirred at room temperature for 4 hours. The reaction mixture was extracted with ethyl acetate twice. The mixed organic phase was washed with a brine, dried over Na₂SO₄, filtered, and concentrated under reduced pressure.

The residue was purified by silica gel column chromatography (SiO₂, EtOAc), so as to provide the target compound 1-4 (356 mg, 99% yield) as a white solid substance (mp: 125-126° C., Rf=0.15 (CHCl₃/MeOH=20/1).

### Data of Compound 1-4:

¹H NMR (400 MHz, CDCl₃) δ 3.88 (s, 3H), 3.95 (s, 3H), 5.54 (br-s, 2H), 6.08-6.10 (m, 1H), 6.60 (d, 1H), 6.65-6.67 (m, 1H), 6.73 (br-s, 1H), 7.13 (d, 1H), 7.71 (s, 1H);

¹³C NMR (100 MHz, CDCl₃) δ 36.6 (2C), 105.1, 107.4, 111.9, 119.8, 121.6, 122.3, 125.6, 128.4, 159.5, 163.3;

IR (ATR) ν 3325, 1636, 1574, 1462, 1417, 1319, 1261, 1115 cm⁻¹

### Example 29

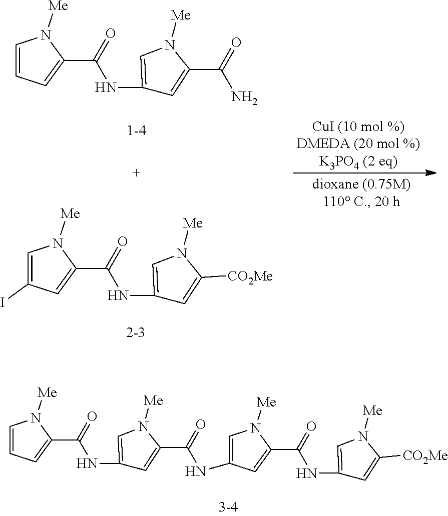 [SMILES: Cn1cc(NC(=O)c2cccn2C)cc1C(N)=O ; COC(=O)c1cc(NC(=O)c2cc(I)cn2C)cn1C ; COC(=O)c1cc(NC(=O)c2cc(NC(=O)c3cc(NC(=O)c4cccn4C)cn3C)cn2C)cn1C]

Under an argon atmosphere, the compound 2-3 (193.6 mg, 0.5 mmol), the compound 1-4 (123.1 mg, 0.5 mmol), CuI (9.5 mg, 0.05 mmol), and K₃PO₄ (212.3 mg, 1.0 mmol) were dissolved in dioxane (0.5 mL). N,N′-dimethylethylenediamine (10.8 μL, 0.1 mmol) was added to the reaction product mixture, which was stirred at 110° C. for 20 hours. The resulting brown suspension was cooled to room temperature, filtered with Celite, and concentrated under reduced pressure.

The residue was purified by silica gel column chromatography (SiO₂, n-hexane/EtOAc=1/2.5), so as to provide the target compound 3-4 (108.7 mg, 43% yield) as a yellow solid substance.

The compound 3-4 is a known compound (Journal of the American Chemical Society 2000, vol. 122, 6382).

### [Synthesis of Py-Py-Py]

### Synthesis Example 12: Synthesis of Compound 4

### Synthesis Example 12-1: Synthesis of Compound 5

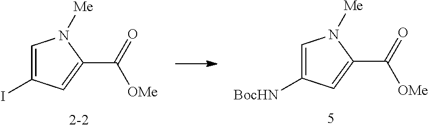 [SMILES: COC(=O)c1cc(I)cn1C ; COC(=O)c1cc(NC(=O)OC(C)(C)C)cn1C]

Under an argon atmosphere, the compound 2-2 (795 mg, 3 mmol), BocNH₂ (1.40 g, 12 mmol), CuI (28.6 mg, 0.15 mmol), and K₃PO₄ (955 mg, 4.5 mmol) were dissolved in dioxane (10 mL). N,N′-dimethylethylenediamine (67.4 mL, 0.6 mmol) was added to the reaction product mixture, which was stirred at 110° C. for 20 hours. The resulting suspension was cooled to room temperature, filtered with Celite, and concentrated under reduced pressure.

The residue was purified by silica gel column chromatography (SiO₂, n-hexane/EtOAc=6/1), so as to provide the target compound 5 (686.3 mg, 90% yield) as a white solid substance.

The compound 5 is a known compound (Journal of Organic Chemistry 2004, vol. 69, 8151).

### Synthesis Example 12-2: Synthesis of Compound 4

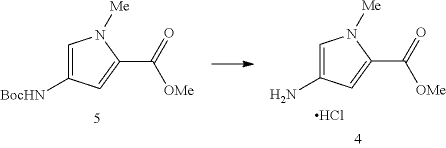 [SMILES: COC(=O)c1cc(NC(=O)OC(C)(C)C)cn1C ; COC(=O)c1cc(N)cn1C ; Cl]

The compound 5 (680 mg, 2.7 mmol) was dissolved in ether (13.5 mL) at 0° C., to which 4 N HCl/dioxane (13.5 mL) was added at the same temperature. The reaction mixture was stirred at room temperature for 20 hours, to which ether was added. The precipitate was collected by filtration, and the resulting filtered material was washed with ether, so as to provide the compound 4 (504.1 mg, 98% yield) as a white solid substance.

The compound 4 is a known compound (Journal of the American Chemical Society 1996, vol. 118, 6141).

### Reference Example 1

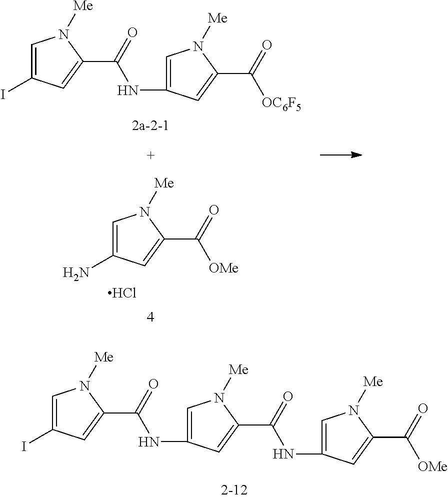

A solution of the compound 2a-2-1 (520 mg, 0.96 mmol), DMAP (29.3 mg, 0.24 mmol), and N,N-diisopropylethylamine (DIPEA) (0.33 mL, 1.92 mmol) in DMF (2 mL) was stirred, to which the compound 4 (247 mg, 1.30 mmol) was added, and the reaction mixed liquid was stirred at room temperature for 16 hours. EtOAc was added to the reaction mixed liquid for diluting, and the resulting mixture was washed with water, a 1 N HCl aqueous solution, a saturated NaHCO₃ aqueous solution, and a brine. The organic phase was dried over Na₂SO₄, and concentrated under reduced pressure.

The residue was purified by silica gel column chromatography (n-hexane/EtOAc=2/1 to 1/2), so as to provide the target compound 2-12 (479.2 mg, 98% yield) as a white solid substance.

### Data of Compound 2-12:

Melting point (mp) 109-110° C.;

¹H NMR (400 MHz, CDCl₃) δ 3.82 (s, 3H), 3.90 (s, 3H), 3.93 (s, 3H), 3.96 (s, 3H), 6.67 (d, J=1.6 Hz, 1H), 6.72 (d, J=1.6 Hz, 1H), 6.73 (d, J=2.0 Hz, 1H), 6.81 (s, 1H), 7.11 (s, 1H), 7.41 (d, J=1.6 Hz, 1H), 7.52 (s, 2H);

¹³C NMR (100 MHz, CDCl₃) δ 36.7, 36.8, 36.9, 51.2, 58.2, 103.7, 108.2, 118.6, 119.4, 119.8, 120.9, 121.1, 121.5, 123.2, 127.4, 132.8, 158.1, 158.8, 161.5;

IR (ATR) ν 3297, 2360, 1636, 1541, 1438, 1398, 1247, 1203, 1110 cm⁻¹;

HRMS (ESI-TOF) m/z: [M+Na]⁺ Calcd. for C₁₉H₂₀1 N₅NaO₄⁺ 532.0452; Found 532.0459.

According to the results of the examples described above, any of PIP sequences up to tetramer units can be synthesized in principle by the production method of the present invention.

In the description herein, the following inventions are described.

<1> A method for producing a nitrogen-containing aromatic amide, including reacting a compound 1 represented by the general formula (1) and a compound 2 represented by the general formula (2) in the presence of a transition metal catalyst and a base, so as to provide a compound 3 represented by the general formula (3).

<2> The method for producing a nitrogen-containing aromatic amide according to the item <1>, wherein the compound 1 is a compound 1′ represented by the general formula (1′), and the compound 2 is a compound 2′ represented by the general formula (2′).

<3> The method for producing a nitrogen-containing aromatic amide according to the item <1>, wherein the compound 1 is a compound 1′ represented by the general formula (1′), and the compound 2 is a compound 2″ represented by the general formula (2″).

<4> The method for producing a nitrogen-containing aromatic amide according to the item <1>, wherein the compound 1 is a compound 1″ represented by the general formula (1″), and the compound 2 is a compound 2′ represented by the general formula (2′).

<5> The method for producing a nitrogen-containing aromatic amide according to the item <1>, wherein the compound 1 is a compound 1″ represented by the general formula (1″), and the compound 2 is a compound 2″ represented by the general formula (2″).

<6> A method for producing a pyrrole-imidazole polyamide, including using the compound 3 obtained by the production method according to any one of the items <1> to <5>.

<7> A method for producing a compound, including:

reacting a compound represented by the general formula (2a-1) and a halogenating agent, so as to provide a compound 2a-2 represented by the general formula (2a-2), and

reacting the compound 2a-2 represented by the general formula (2a-2) and an alcohol having from 1 to 6 carbon atoms in the presence of a base, so as to provide a compound 2a represented by the general formula (2a).

<8> A method for producing a compound, including:

reacting a compound 2a-3 represented by the general formula (2a-3) and a halogenating agent, so as to provide a compound 2a-4 represented by the general formula (2a-4), and

substituting a protective group of the compound represented by the general formula (2a-4) with a hydrogen atom, so as to provide a compound 2a represented by the general formula (2a).

<9> A compound represented by the general formula (2aa), the general formula (2ab), the general formula (2ac), or the general formula (2ad).

1. A method for producing a nitrogen-containing aromatic amide, comprising reacting a compound 1 represented by the general formula (1): 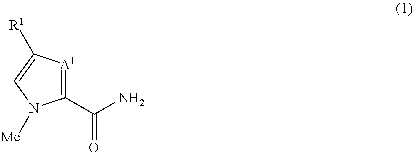
wherein R¹ represents a hydrogen atom or a substituent; and A¹ represents N or CH, and
a compound 2 represented by the general formula (2): 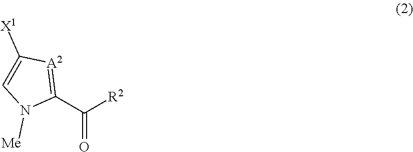
wherein A² represents N or CH; X¹ represents a halogen atom; and R² represents an alkyloxy group having from 1 to 6 carbon atoms or a group represented by the general formula (A): *—NHR^{a}, wherein R^{a} represents a substituent; and * represents a bonding site,
in the presence of a transition metal catalyst and a base, so as to provide a compound 3 represented by the general formula (3): 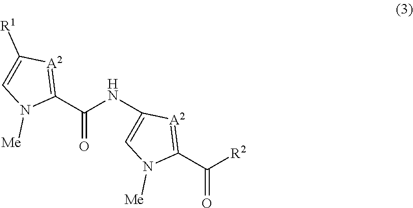
wherein R¹, A¹, A², and R² have the same meanings as in the general formula (1) and the general formula (2).

2. The method for producing a nitrogen-containing aromatic amide according to claim 1, wherein the compound 2 is a compound represented by the general formula (2a): 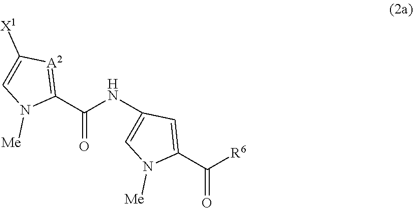
wherein X¹ represents a halogen atom; A² represents N or CH; and R⁶ represents an alkyloxy group having from 1 to 6 carbon atoms, and
the method further comprises:
reacting a compound represented by the general formula (2a-1): 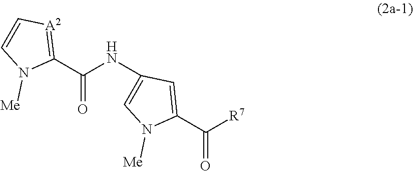
wherein A² represents N or CH; and R⁷ represents a fluorinated aryloxy group, a fluorinated alkyloxy group having from 1 to 6 carbon atoms, or a nitrophenyloxy group, and
a halogenating agent,
so as to provide a compound represented by the general formula (2a-2): 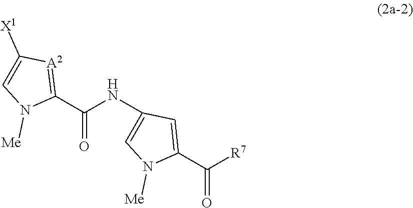
wherein X¹ represents a halogen atom; and A² and R⁷ have the same meanings as in the general formula (2a-1); and
reacting the compound represented by the general formula (2a-2) and an alcohol having from 1 to 6 carbon atoms in the presence of a base, so as to provide the compound represented by the general formula (2a).

3. The method for producing a nitrogen-containing aromatic amide according to claim 2, wherein R⁷ represents a pentafluorophenyloxy group.

4. The method for producing a nitrogen-containing aromatic amide according to claim 1, wherein the compound 2 is a compound represented by the general formula (2a): 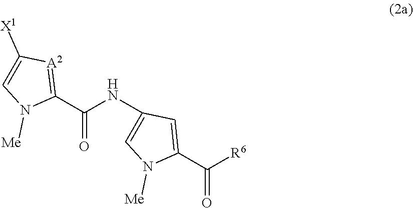
wherein X¹ represents a halogen atom; A² represents N or CH; and R⁶ represents an alkyloxy group having from 1 to 6 carbon atoms, and
the method further comprises:
reacting a compound represented by the general formula (2a-3): 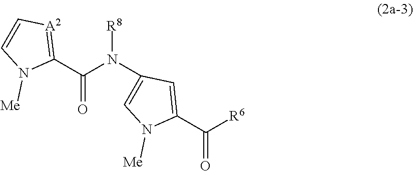
wherein A² represents N or CH; R⁶ represents an alkyloxy group having from 1 to 6 carbon atoms; and R⁸ represents a protective group selected from the group consisting of a nosyl group, a trimethylsilylethanesulfonyl group, a methanesulfonyl group, a trifluoroacetyl group, and a trifluoromethanesulfonyl group, and
a halogenating agent,
so as to provide a compound represented by the general formula (2a-4): 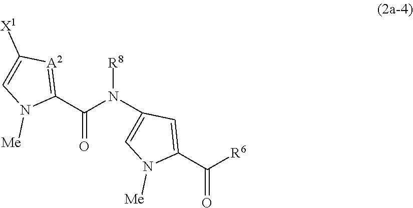
wherein X¹ represents a halogen atom; and A², R⁶ and R⁸ have the same meanings as in the general formula (2a-3), and
substituting the protective group of the compound represented by the general formula (2a-4) with a hydrogen atom, so as to provide the compound represented by the general formula (2a).

5. The method for producing a nitrogen-containing aromatic amide according to claim 2, wherein the halogenating agent is an N-halosuccinimide.

6. The method for producing a nitrogen-containing aromatic amide according to claim 1, wherein X¹ represents an iodine atom.

7. The method for producing a nitrogen-containing aromatic amide according to claim 1, wherein the transition metal catalyst is a copper catalyst.

8. The method for producing a nitrogen-containing aromatic amide according to claim 1,
wherein R¹ represents a hydrogen atom, a BocHN group, an AcHN group, or a group represented by the general formula (B): 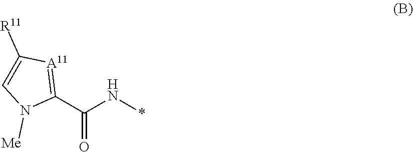
wherein R¹¹ represents a hydrogen atom, a BocHN group, an AcHN group, or a group represented by the general formula (B′): 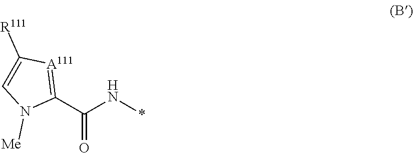
wherein R¹¹¹ represents a hydrogen atom, a BocHN group or an AcHN group; A¹¹¹ represents N or CH; and * represents a bonding site;
A¹¹ represents N or CH; and
* represents a bonding site.

9. The method for producing a nitrogen-containing aromatic amide according to claim 1,
wherein R^{a} represents an alkyl group having from 1 to 6 carbon atoms, an aralkyl group having from 7 to 20 carbon atoms, an aminoalkyl group having from 1 to 6 carbon atoms, or a group represented by the general formula (C): 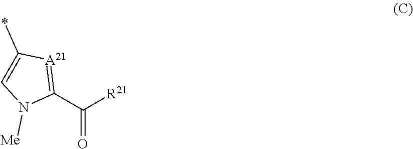
wherein A²¹ represents N or CH; and R²¹ represents an alkyloxy group having from 1 to 6 carbon atoms or a group represented by the general formula (A′): *—NHR^{a′}, wherein R^{a′} represents a substituent; and * represents a bonding site.

10. A method for producing a pyrrole-imidazole polyamide, comprising using the compound 3 obtained by the production method according to claim 1.

11. A compound represented by the general formula (2aa): 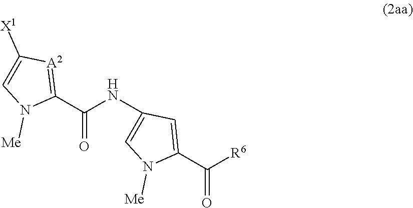
wherein A² represents N or CH; X¹ represents a halogen atom; and R⁶ represents an alkyloxy group having from 1 to 6 carbon atoms,
the general formula (2ab): 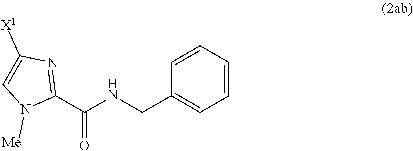
wherein X¹ represents a halogen atom,
the general formula (2ac): 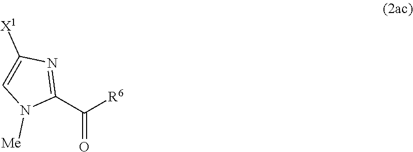
wherein X¹ represents a halogen atom; and R⁶ represents an alkyloxy group having from 1 to 6 carbon atoms, or
the general formula (2ad): 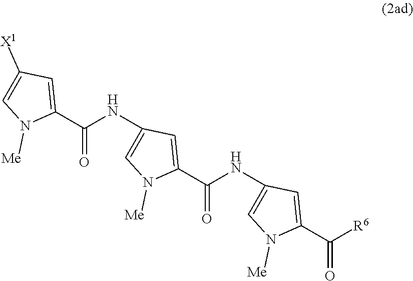
wherein X¹ represents a halogen atom; and R⁶ represents an alkyloxy group having from 1 to 6 carbon atoms.

12. The method for producing a nitrogen-containing aromatic amide according to claim 4, wherein the halogenating agent is an N-halosuccinimide.

13. The method for producing a nitrogen-containing aromatic amide according to claim 4, wherein the transition metal catalyst is a copper catalyst.
